(19) United States
(12) Patent Application Publication
(10) Pub. No.: US 2013/0108688 A1
(43) Pub. Date: May 2, 2013

(54) Delivery of H2 Antagonists

(71) Applicant: Trustees of Boston University, Boston, MA (US)

(72) Inventors: Thomas E. Van Dyke, West Roxbury, MA (US); Michael Holick, Sudbury, MA (US); Alpdogan Kantarci, Brighton, MA (US); Hatice Hasturk, Brighton, MA (US)

(73) Assignee: Trustees of Boston University, Boston, MA (US)

(21) Appl. No.: 13/705,471

(22) Filed: Dec. 5, 2012

Related U.S. Application Data
(63) Continuation of application No. 13/027,437, filed on Feb. 15, 2011.
Application No. 13/027,437 is a continuation of application No. 11/793,884, filed on Jun. 21, 2007, now abandoned.
(60) Provisional application No. 60/639,892, filed on Dec. 29, 2004.

(51) Int. Cl. A61K 9/00

(52) U.S. Cl. CPC A61K 9/0002
USPC 424/450; 514/400; 514/370; 514/365; 514/471

(57) ABSTRACT

The present invention provides improved systems and methods for the local delivery of H2 antagonists. The inventive methods include topical administration of an effective amount of a H2 antagonist encapsulated in liposomes. In certain embodiments, the H2 antagonist, for example, Cimetidine, is encapsulated into paucilamellar liposomes, such as NOVASOME® microvesicles. Also provided are pharmaceutical compositions comprising liposome-encapsulated H2 antagonists. The methods and compositions of the present invention may be used to treat any disease state or condition where local administration of H2 antagonists is beneficial.

### RELATED APPLICATIONS

The present application is a continuation of Ser. No. 13/027,437, which is a continuation of U.S. Non-Provisional Application No. Ser. No. 11/793,884, filed Jun. 21, 2007, which is a national phase application under 35 U.S.C. § 371 of International Application No, PCT/US05/046280, filed Dec. 20, 2005, which claims priority to U.S. Provisional Application No. 60/639,892, filed Dec. 29, 2004. The entirety of each of these priority applications is incorporated herein by reference.

### BACKGROUND OF THE INVENTION

Periodontal disease, ranging from gingivitis to more severe forms of periodontitis, remains a significant health problem and is a major cause of tooth loss in adults both in the United States and throughout the world (E. Reich and K. Hiller, Comm. Dent. Oral Epidem., 1993, 21: 379; J. Angelillo et al., Comm. Dent. Oral Epidem., 1996, 24: 336; H. Murray et al., Int. Dent, J., 1997, 47: 3-8; R. C. Oliver et al., J. Periodontol., 1998, 69: 269-278; G. Ong, Int Dental J., 1998, 48: 233-238; I. Haddad et al., Dental J., 1999, 49: 343-346; E. F. Corbet et al., Periodontology, 2000, 29: 122-152; A. Sheiham et al., Periodontology, 2000, 29: 104-121; I. Chestnutt et al., J. Dentist., 2000, 28: 295-297; U. M. Irfan et al., J. Int. Acad, Periodontol., 2001, 3: 14-21). It has been estimated that periodontal disease affects 20 to 30% of all adults in the industrialized world, In the U.S. alone, roughly 67 million adults are believed to be affected (J. M, Albandar et al., J. Periodontol., 1999, 70: 13-29). This prevalence makes periodontal disease one of the most common chronic infectious diseases afflicting adults. Furthermore, periodontal disease has implications beyond the deleterious effects on oral tissues and structural integrity, and represents a potential risk factor for increased morbidity and mortality for several systemic conditions including cardiovascular diseases, pregnancy complications and diabetes (R. C. Page et al., Ann. Periodontol., 1998, 3: 108-120; R.I. Garcia et al., Ann. Periodontol., 1998, 3: 339-349).

Out of the hundreds of bacterial species present in the oral cavity, only a small number are involved in the etiology of periodontal disease (S. S. Socransky and A. D. Haffajee, Periodontal., 2002, 28: 12-55). The biofilm may contain bacteria, such as Porphyromonas gingivalis, Bacteroides forsythus, and Treponema denticola, the presence of which has been found to be strikingly related to clinical features of periodontal disease, in particular pocket depth and bleeding on probing (S. S. Socransky et al., J. Clin. Periodontol., 1998, 25: 134-144). Some of these pathogenic organisms can invade periodontal tissues, dentinal tubules, as well as other areas of the oral cavity.

Conventional periodontal therapy has emphasized mechanical removal of soft and hard accretions of bacteria from the root surface via use of dental instruments placed into the gingival crevice. However, scaling and root planning is often only partially effective in removing these accretions. Even though bacteria removal will reduce tissue destruction, some patients do not respond predictably to such a reduction in bacteria (I. Brook, Gen. Dent., 2003, 51: 424-428; R. C. Page, J. Periodontal. Res., 1991, 26: 230-252). Moreover, even for easily accessible areas, bacteria removal is only transient and the bacteria generally re-colonize the root surface. Thus, in addition to bacterial control, a modern approach to the treatment of periodontal disease should aid in the prevention of the disease or enhance clinical therapeutic responses in susceptible hosts (R. C. Page, J. Periodontal. Res., 1991, 26: 230-242; M. S. Reddy et al., Ann. Periodontol., 2003, 8: 12-37; T. E. Van Dyke and C. N. Serhan, J. Dent. Res., 2003, 82: 82-90).

Scientists have made numerous efforts to study the initiation and contributing factors of periodontal tissue destruction, in order to identify and develop new ways to treat and/or prevent it. Research over the last few decades has shown that the host plays an important role in the initiation and progression of periodontal disease (G. J. Seymour, J. Clin. Periodontol., 1991, 18: 421-426; I. Brook, Gen. Dent., 2003, 51: 424-428). When the virulent bacteria begin to flourish in the periodontal region, toxic and pathogenic products are released and induce an inflammatory response. Inflammatory cells, including polymorphonuclear leukocytes, monocytes, lymphocytes, macrophages, mast cells, and plasma cells, are recruited to infiltrate the periodontium and clear the area of the pathogenic organisms (L. Graham, Gen. Dent., 2003, 51: 570-578). Mast cells play an important role in the early propagation of the inflammatory response due to their cytoplasmic granules that contain substances such as histamine, slow-reacting substance of anaphylaxis, heparin, eosinophil chemotactic factor of anaphylaxis, and bradykinin, all of which are released in gingival tissues. One of the most important mast cell-derived mediators of inflammation, histamine, has a suppressive effect on a variety of neutrophil, macrophage and monocyte functions involved in the protective host tissue response against plaque bacteria and their products. These suppressive effects are mediated via binding to H2 receptors on the cell surface (N. Hirasawa et al., Inflammation, 1991, 15: 117-126; H.J. Nielsen et al., Arch. Surg., 1994, 129: 309-315)

The effects of histamine can generally be counteracted by antihistamine drugs including histamine-2 receptor antagonists (H2 antagonists). Methods for treating periodontal disease have been disclosed that involve topical administration of H2 antagonists to mucosal tissues (e.g., gingival mucosa) of the oral cavity (U.S. Pat. Nos. 5,294,433 and 5,364,616). Systemic administration of H2 antagonists for the treatment of bone disease, including bone loss resulting from periodontal disease, has also been described (see PCT application No. WO 89/04178). However, systemic delivery (e.g., oral or intramuscular) typically does not provide a sufficient concentration of H2 antagonists over an extended period of time to the gingival crevice area; and topical application of such agents in solution has been found to lead to only weak absorption of H2 antagonists. Thus, there remains a need for novel methods for preventing and treating periodontal diseases.

### SUMMARY OF THE INVENTION

The present invention relates to new systems and strategies for the delivery of H2 antagonists. More specifically, the present invention provides compositions and methods that allow for improved topical administration of H2 antagonists. The compositions and methods of the present invention can be used for the treatment and/or prevention of any disease state or condition for which local application of H2 antagonists is beneficial.

In particular, in one aspect, the present invention provides a liposomal composition comprising a H2 antagonist and liposome, wherein the H2 antagonist is encapsulated in the liposome. In some embodiments, the H2 antagonist comprises a compound selected from the group consisting of cimetidine, famotidine, nizatidine, and combinations thereof The H2 antagonist may be encapsulated in a liposome selected from the group consisting of unilamellar liposome, multilamellar liposome and paucilamellar liposome. In certain embodiments, the liposome is a paucilamellar liposome.

In another aspect, the present invention provides a pharmaceutical composition comprising an effective amount of a liposome-encapsulated H2 antagonist and at least one physiologically acceptable excipient. In certain embodiments, the H2 antagonist and liposome are as described above. The pharmaceutical composition may be in a form selected from the group consisting of: solutions, suspensions, dispersions, ointments, creams, pastes, gels, powders, lozenges, salve, chewing gums, sprays, pastilles, sachets, aerosols, tablets, capsules, and transdermal patches. For example, the pharmaceutical composition may be in a form selected from the group consisting of toothpastes, chewing gums, mouth sprays, mouthwashes, tooth powders, toothpicks, and dental floss.

In certain embodiments, pharmaceutical compositions of the present invention further comprise at least one additional therapeutic agent. For example, the additional therapeutic agent may comprise an antimicrobial compound, a non-steroidal anti-inflammatory compound or a H1 antagonist.

In yet another aspect, the present invention provides a method for delivering a H2 antagonist to a subject, the method comprising a step of administering to the subject a pharmaceutical composition, as disclosed herein. The step of administering may comprise topically administering the pharmaceutical composition, for example, to a human subject's skin or mucosa. In certain embodiments, the subject is suffering from or is susceptible to a condition for which local delivery of a H2 antagonist is beneficial. For example, the subject may be suffering from or may be susceptible to a condition affecting the oral cavity, such as aphthous ulcers or herpes stomasis, or a periodontal disease, e.g., gingivitis or periodontitis. Alternatively or additionally, the subject may be suffering from or may be susceptible to a systemic condition associated with periodontal disease, such as cardiovascular disease, pregnancy complications or diabetes. In certain embodiments, the subject is suffering from or is susceptible to a condition affecting the skin or mucosa, such as psoriasis, atopic eczema, urticaria, allergic reaction, warts, or burn itch.

In still another aspect, the present invention provides a method for preventing or treating periodontal disease, e.g., gingivitis or periodontitis, in a subject, the method comprising a step of administering to the subject an effective amount of a liposome-encapsulated H2 antagonist. In certain embodiments, the H2 antagonist and liposome are as described above. In some embodiments, the liposome-encapsulated H2 antagonist is topically administered to the subject's oral cavity. The subject may be suffering from or may be susceptible to a condition associated with periodontal disease, e.g., cardiovascular disease, pregnancy complications or diabetes.

These and other objects, advantages and features of the present invention will become apparent to those of ordinary skill in the art having read the following detailed description.

### BRIEF DESCRIPTION OF THE DRAWING

FIG. 1 presents pictures of the mandibles of rabbits treated, as described in the Examples section below, by ligature alone (Group A); ligature+P. gingivalis (Group B); ligature+NOVASOME® (Group C); ligature+P. gingivalis+NOVASOME® preparation comprising 0.1 μg/mL of cimetidine (Group D); ligature+P. gingivalis+NOVASOME® preparation comprising 1.0 μg/mL of cimetidine (Group E); or ligature+P. gingivalis+NOVASOME® preparation comprising 10 μg/mL of cimetidine (Group F). Each panel (A to F) contains 4 sets of pictures (each one showing gingival tissue and defleshed bone specimens from buccal and lingual sites). Arrows depict the soft and hard tissue changes observed in Groups B and C of animals.

FIG. 2 presents, on a graph, the results of a quantitative analysis of alveolar bone levels of defleshed bone specimens as a function of localization in the oral cavity (i.e., buccal interproximal, lingual interproximal, buccal crestal and lingual crestal) and as a function of treatment received by the different animal groups (i.e., ligature alone (A), ligature+P. gingivalis(B), ligature+NOVASOME® (C), ligature+P. gingivalis+NOVASOME® preparation comprising 0.1 μg/mL (D), 1.0 μg/mL (E) or 10 μg/mL (F) of cimetidine).

FIG. 3 presents the results of a radiographic analysis of bone and other hard tissue components. The pictures show the radiography of specimens that have received ligature alone (A), ligature+P. gingivalis (B), ligature+NOVASOME® (C), ligature+P. gingivalis+NOVASOME® preparation comprising 0.1 μg/mL (D), 1.0 μg/mL (E) or 10 μg/mL (F) of cimetidine. Bone loss (which is clearly visible and indicated by arrows in B and C) is prevented by topical application of Cimetidine (as indicated by arrows in D, E and F, where alveolar bone is at the same level as in animals that have received the ligature application alone, A). The graph presents the percentage of bone loss as calculated by Bjorn technique (see Examples) as a function of treatment received by the different groups of animals.

FIG. 4 presents a set of histological pictures of H&E stained sections of the ligated sites showing the changes undergone in response to different treatments (i.e., ligature alone (A), ligature+P. gingivalis (B), ligature+NOVASOME® (C), ligature+P. gingivalis+NOVASOME® preparation comprising 0.1 μg/mL (D), 1.0 μg/mL (E) or 10 μg/mL (F) of cimetidine). Inflammatory cells are indicated by the sign *, and bone resorption is depicted by a black arrow. Ligature placement alone led to increased numbers of inflammatory cells while neither bone loss nor any osteoclastic activity were visible (Panel A). Local P. gingivalis administration in addition to ligature placement led to significant bone resorption and increased inflammation (Panel B). Liposome alone did not have any preventive or aggravating effect on the development of periodontitis (Panel C), while all three doses of topical Cimetidine applications were found to prevent both bone loss and inflammatory changes (Panels D, E and F).

FIG. 5 presents a set of histological pictures of TRAP stained sections of the ligated sites showing the changes undergone in response to different treatments (i.e., ligature alone (A), ligature+P. gingivalis (B), ligature+NOVASOME® (C), ligature+P. gingivalis+NOVASOME® preparation comprising 0.1 μg/mL (D), 1.0 μg/mL (E) or 10 μg/mL (F) of cimetidine). Ligation alone did not lead to any increase in osteoclast numbers (Panel A). The alveolar bone borders were found to be extremely ruffled with increased numbers of irregular shaped Howship's resorptive lacunae presenting osteoclastic activity (Panel B). In the vehicle control group, liposomes alone were not found to prevent the osteoclastic activity (Panel C). However, in the three Cimetidine groups (Panels D, E and F), osteoclastic cells were either unidentifiable or at few numbers.

FIG. 6 presents a series of three graphs showing the results of a histomorphometrical analysis performed for the different animal groups (i.e., animals that have received ligature alone (A), ligature+P. gingivalis (B), ligature+NOVASOME® (C), ligature+P. gingivalis+NOVASOME® preparation comprising 0.1 μg/mL (D), 1.0 μg/mL (E) or 10 μg/mL (F) of cimetidine). The graph in FIG. 6A presents the mean value (±standard deviation) of the linear distances (i.e., the distances from the epithelium to the alveolar crest border) measured at three different levels, the tip, the middle, and the base of the crest and expressed as the ratio between the ligated and non-ligated sites. The ligated sites in Groups B and C showed significant increased (p<0.05) distances compared to the Cimetidine-treated groups (Groups D, E and F). The graph in FIG. 6B presents the areas expressed as the proportion of the total area at ligated to the non-ligated aspects of the teeth. The total area as well as the area of ligated side of the alveolar crest was significantly reduced in the control and vehicle groups (p<0.05). The graph in FIG. 6C presents the number of osteoclasts at the apical, middle, and coronal thirds of the root. Groups B and C exhibited markedly increased numbers of osteoclasts at all three levels with statistically significant values (p<0.05) whereas the Cimetidine groups showed comparable, non-significant values at the tip, middle and the base of the crest (p<0.05).

### DEFINITIONS

For purpose of convenience, definitions of a variety of terms used throughout the specification are presented below.

As used herein, the term “liposome” refers to unilamellar vesicles or multilamellar vesicles such as those described in U.S. Pat. No. 4,753,788, which is incorporated herein by reference in its entirety. General information about liposomes can be found in a variety of textbooks including, for example, “Liposomes”, M. J. Ostro (Ed.), 1987, Marcel Dekker; “Liposome Drug Delivery Systems”, G. V. Betageri, S. A. Jenkins and D. L. Parson (Eds.), 1993, CRC Press; “Liposomes Methods and Protocols (Methods in Molecular Biology)” S. C. Basu and M. Basu (Eds.), 2002, Humana Press.

The terms “unilamellar liposomes”, “unilamellar vesicles” and “single lamellar vesicles” are used herein interchangeably. They refer to substantially spherical vesicles comprising one lipid bilayer membrane which defines a single closed aqueous compartment. The bilayer membrane is composed of two layers of lipids; an inner layer and an outer layer. The lipid molecules in the outer layer are oriented with their hydrophilic head portions toward the external aqueous environment and their hydrophobic tails pointed toward the interior of the liposome. The inner layer lays directly beneath the outer layer; and the lipids in the outer layer are oriented with their heads facing the aqueous interior of the liposomes and their tails toward the tails of the outer layer of lipid.

The terms “multilamellar liposomes”, “multilamellar vesicles”, and “multiple lamellar vesicles” are used herein interchangeably. They refer to substantially spherical vesicles composed of two or more lipid bilayer membranes, which membranes define more than one closed aqueous compartment. The membranes are concentrically arranged so that the different membranes are separated by aqueous compartments.

The terms “paucilamellar liposomes” and “paucilamellar vesicles” are used herein interchangeably. They refer to substantially spherical vesicles composed of about 2 to about 10 lipid bilayer membranes delimiting a large, unstructured (i.e., amorphous) central aqueous volume.

The terms “encapsulated” and “entrapped” are used herein interchangeably. They refer to the incorporation or association of a substance or molecule (e.g., a drug) in or with a liposome. The substance or molecule may be associated with the lipid bilayer or present in the aqueous interior of the liposome, or both.

The terms “excipient”, “counterion” and “counterion excipient” are used herein interchangeably. They refer to a chemical entity that can initiate or facilitate substance loading in the liposome. Alternatively or additionally, they refer to a chemical entity that can initiate or facilitate precipitation of the substance in the aqueous interior of the liposome. Examples of excipients include, but are not limited to, the acid, sodium or ammonium forms of monovalent anions such as chloride, acetate, lactobionate and formate; divalent anions such as aspartate, succinate and sulfate; and trivalent ions such as citrate and phosphate.

The term “phospholipid” refers to any one phospholipid or combination of phospholipids capable of forming liposomes. Phosphatidylcholines (PCs), including those obtained from egg, soy beans or other plant sources or those that are partially or wholly synthetic, or of variable lipid chain length and unsaturation are suitable for use in the present invention. Synthetic, semisynthetic and natural product phosphatidylcholines including, but not limited to, diastearoylphosphatidylcholine (DSPC), hydrogenated soy phosphatidylcholine (HSPC), soy phosphatidylcholine (soy PC), egg phosphatidulcholine (egg PC), hydrogenated egg phosphatidylcholine (HEPC), dipalmitoylphosphatidylcholine (DPPC), and dimyristoyl-phosphatidylcholine (DMPC) are suitable phosphatidylcholines for use in the compositions and methods of the present invention. These phospholipids are commercially available.

Other suitable phospholipids include phosphatidylglycerols (PGs) and phosphatic acids (PAs). Examples of suitable phosphotidylglycerols include, but are not limited to, dimyristoylphosphatidylglycerol (DMPG), dilauryl-phosphatidylglycerol (DLPG), dipalmitoyl-phosphatidylglycerol (DPPG), and distearoylphosphatidylglycerol (DSPG). Non-limiting examples of suitable phosphatic acids include dimyristoylphosphatidic acid (DMPA), distearoylphosphatidic acid (DSPA), dilaurylphosphatidic acid (DLPA), and dipalmitoyl-phosphatidic acid (DPPA). Other suitable phospholipids include phosphatidylethanolamines, phosphatidylinositols, and phosphatic acids containing lauric, myristic, stearoyl, and palmitic acid chains.

The terms “local” and “topical”, when used to characterize the delivery, administration or application of a composition of the present invention, is meant to specify that the composition is delivered, administered or applied directly to the site of interest (e.g., in the oral cavity for an oral disorder such as periodontal disease) for a localized effect. In certain embodiments, local or topical administration is effected without any significant absorption of components of the composition into the subject's blood stream.

As used herein, the term “effective amount” refers to any amount of a molecule, agent, factor, or composition that is sufficient to fulfill its intended purpose(s) (e.g., the purpose may be to treat or prevent periodontal disease) when the molecule, agent, factor or composition is delivered, administered or applied locally.

As used herein, the term “physiologically acceptable carrier or excipient” refers to a carrier medium or excipient which does not interfere with the effectiveness of the biological activity of the active ingredients and which is not excessively toxic to the host at the concentrations at which it is administered. The term includes solvents, dispersion media, coatings, antibacterial and antifungal agents, isotonic agents, absorption delaying agents, and the like. The use of such media and agents for the formulation of pharmaceutically active substances is well-known in the art (see, for example, “Remington's Pharmaceutical Sciences”, E. W. Martin, 18^{th} Ed., 1990, Mack Publishing Co.: Easton, Pa., which is incorporated herein by reference in its entirety).

The terms “individual” and “subject” are used herein interchangeably. They refer to a higher vertebrate, preferably a human or another mammal (e.g., a mouse, rat, rabbit, monkey, dog, cat, pig, cow, horse, and the like), that may or may not have a disease state or condition for which local administration of a H2 antagonist is beneficial.

The term “prevention” is used herein to characterize a method that is aimed at delaying or preventing the onset of a disease state or condition. The treatment is administered prior to the onset of the condition, for a prophylactic action. The term “treatment” is used herein to characterize a method that is aimed at (1) slowing down or stopping the progression, aggravation, or deterioration of the symptoms of a clinical condition; (2) bringing about ameliorations of the symptoms of the condition; and/or (4) curing the condition. The treatment is administered after initiation of the condition for a therapeutic action.

### DETAILED DESCRIPTION OF CERTAIN PREFERRED EMBODIMENTS

As mentioned above, the present invention relates to new systems and strategies for the delivery of H2 antagonists. More specifically, the present invention provides compositions and methods that allow for improved topical administration of H2 antagonists.

### I. H2 Antagonists

Histamine 2 receptor antagonists (H2 antagonists) are compounds that block H2 receptors. In certain embodiments, H2 antagonists to be used in the compositions and methods of the present invention exhibit a selective activity: they block E2 receptors but do not have a meaningful activity in blocking histamine 1 receptors. Histamine stimulates the contraction of smooth muscle from various organs, such as the gut and bronchi; this effect can be suppressed by low concentration of mepyramine—a typical antihistamine drug. The pharmacological receptors involved in these mepyramine-sensitive histamine responses have been defined as H1 receptors (A. S. F Ash and O. Schild, Brit. J. Pharmacol. Chemother., 1966, 27: 427-439). Histamine also stimulates the secretion of acid by the stomach, increases the heart rate, and inhibits contractions in the rat uterus; these actions are not antagonized by mepyramine and related drugs. H2 antagonists suitable for use in the compositions and methods of the present invention include those that blockade the receptors involved in mepyramine-insensitive histamine responses, and do not significantly blockade the receptors involved in mepyramine-sensitive histamine responses.

H2 antagonists suitable for use in the compositions and methods of the present invention include those compounds found to be H2 antagonists through their performance in classical preclinical screening tests for H2 antagonistic function. Suitable H2 antagonists include compounds which can be demonstrated to function as competitive or non-competitive inhibitors of histamine-mediated effects in those screening models specifically dependent upon H2 receptor function, but lack significant histamine antagonist activity in those screening models dependent upon H1 receptor function. For example, this includes compounds that would be classified, as described by J. W. Black et al., as H2 antagonists if assessed through testing with guinea pig spontaneously beating right atria in vitro assay and the rat gastric acid secretion in vivo assay, but shown to lack in significant H1 antagonist activity trough testing with either the guinea pig ileum contraction in vitro assay or the rat stomach muscle contraction in vivo assay (Nature, 1972, 236: 385-390). In certain embodiments, H2 antagonists to be used in the practice of the present invention demonstrate no significant H1 activity at reasonable dosage levels in the above-mentioned H1 assays. Typically, reasonable dosage level is the lowest dosage level at which 90% inhibition of histamine, or 99% inhibition, is achieved in the above-mentioned H2 assays.

Examples of suitable selective H2 antagonists for use in the compositions and methods of the present invention include compounds meeting the above criteria which are described in U.S. Pat. Nos. 5,294,433 and 5,364,616 and references cited therein (each of these U.S. patents and references is incorporated herein by reference in its entirety).

Specific examples of suitable H2 antagonists include, but are not limited to, Cimetidine (also known as Tagamet or Dyspamet), Famotidine (also known as Pepcid), Nizatidine (also known as Axid), Ranitidine (also known Zantac), and Ranitidine bismuth citrate (also known as Pylorid).

In certain embodiments, Cimetidine is used as H2 antagonist in the compositions and methods of the present invention. Cimetidine (N″-cyano-N-methyl-N′-[2-[[5-methyl-1H-imidazol-4-yl)methyl]thio]ethyl]guanidine) has been described in U.K. Patent No. 1,397,426 and U.S. Pat. Nos. 3,950,233 and 4,024,271 (each of which is incorporated herein by reference in its entirety). Cimetidine is also disclosed in the Merck Index (1989, 11^{th} Ed., p. 354, entry No. 2279) and Physician's Desk Reference (1992, 46^{th} Ed., p 2228).

Cimetidine is used in the treatment of duodenal, gastric, recurrent and stomal ulceration, and reflux esophagitis and in the management of patients who are at high risk from hemorrhage of the upper gastro-intestinal tract (S. A. Alekseenko and S. S. Timoshin, Ter. Arkh. 1999, 71: 23-26). In AIDS patients, Cimetidine administration has been shown to have a significant improvement effect on clinical symptoms of disease (N. H. Brockmeyer et al., Clin. Immunol. Immunopathol., 1988, 48: 50-60). It eliminates histamine inhibitory effects on chemotaxis, phagocytosis, superoxide anion production and the production of TNF-α and IL-12 by macrophages via H2 receptor (G. Hotermans et al., Int. Arch. Allergy Appl. Immunol., 1991, 95: 278-281; H. Ishikura et al., Blood, 1999, 93: 1782-1783; J. A. Majeski and J. W. Alexander, J. Surg. Oncol., 1981, 17: 139-144). Cimetidine has been shown to completely reverse the histamine-mediated increase in IL-α induced IL-6 synthesis (D. MacGlashan Jr., J. Allergy Clin. Immunol., 2003, 112: S53-S59) via activation of H2 receptor (E. Vannier and C.A. Dinarello, J. Clin. Invest., 1993, 92: 281-287). The proposed mechanism of the immunomodulative effects of H2 receptor antagonists was suggested to be mediated through inhibition of suppressor T-lymphocyte activity, an increase in IL-2 production, and an enhancement of natural killer cell activity (W. B. Ershler et al., Clin. Immunol. Immunopathol., 1983, 26: 10-17; K. B. Hahm et al., Scand. J. Gastroenterol., 1995, 30: 265-271). Administration of Cimedidine, 800 mg daily for a period of 7 days to healthy volunteers, led to a decrease of CD8 (cytotoxic/suppressor) lymphocytes along with a corresponding increase in the CD4 (helper/inducer) lymphocytes (N. H. Brockmeyer et al., Klin. Wochenschr., 1989, 67: 26-30; N. H. Brockmeyer et al., J. Invest. Dermatol., 1989, 93: 757-761).

Other specific examples of suitable H2 antagonists include ranitidine (i.e., N-(2-(((5-((dimethylamino)methyl)-2-furanyl)methyl)thio)ethyl)-N′-methyl-2-nitro-1,1-ethenediamine, which is described in U.S. Pat. No. 4,128,658; the Merck Index, 11^{th} Ed., 1989, p. 1291, entry 8126) and Physicians' Desk Reference, 46^{th} Ed., 1992, p. 1063); famotidine (i.e., 3-(((2-((aminoiminomethyl)amino)-4-thiazolyl)methyl)thio)-N-aminosulfonyl) propanimidamide, which is described in U.S. Pat. No. 4,283,408; the Merck Index, 11^{th} Ed., 1989, p. 617, entry No. 3881; and Physicians' Desk Reference, 46^{th} Ed., 1992, p. 1524); nizatidine (i.e., N-(24(2-((dimethylamino)methyl)-4-thiazolyl)methyl)thio)ethyl)-N′-methyl-2-nitro-1,1-ethenediamine, which is described in U.S. Pat. No. 4,375,547; the Merck Index, 11^{th} Ed., 1989, p. 1052, entry No. 6582; and Physicians' Desk Reference, 46^{th} Ed., 1992, p. 1246); and mifentidine (i.e., N-(4-(1H-imidazol-4-yl)phenyl)-N′-(1-methylethyl)methanimidamide, which is disclosed in U.S. Pat. No. 4,386,099; the Merck Index, 11^{th} Ed., 1989, p. 973, entry No. 6108).

H2 antagonists to be used in the compositions and methods of the present invention may be prepared using conventional synthetic methods; or, alternatively, they can be obtained from commercial sources.

As will be appreciated by one of ordinary skill in the art, H2 antagonists suitable for use in the inventive compositions and methods can be under in a free form or a pharmaceutically acceptable salt form. The term “pharmaceutically acceptable salts”, as used herein, refers to salts that are, within the scope of sound medical judgment, suitable for use in contact with the tissues of subjects to be treated without undue toxicity, irritation, allergic response, and the like, commensurate with a reasonable benefit/risk ratio, and effective for their intended use.

The term “salts” refers to the relatively non-toxic inorganic and organic acid addition or base addition salts of H2 antagonists. These salts can be prepared in situ during the final isolation and purification of the compounds or by separately reacting the purified compound in its free form with a suitable organic or inorganic acid or base and isolating the salt thus formed. Acid addition salts can be formed with inorganic acids (e.g., hydrochloric, hydrobromic, sulfuric, nitric, phosphoric acids, and the like) or organic acids (e.g., acetic, propionic, pyruvic, maleic, malonic, succinic, fumaric, tartaric, citric, benzoic, mandelic, methanesulfonic, ethanesulfonic, p-toluenesulfonic, salicylic acids, and the like). Base addition salts can be formed with inorganic bases (e.g., sodium, potassium, lithium, ammonium, calcium, magnesium, zinc, aluminum salts, and the like) or organic salts (e.g., salts of primary, secondary, and tertiary amines, substituted amines including naturally occurring substituted amines, cyclic amines, basic ion exchange resins, polyamine resins, and the like).

### II. Liposomal Preparations of H2 Antagonists

Liposomes suitable for use in the practice of the present invention include any liposomal system that can micro-encapsulate one or more H2 antagonists and act as a vehicle for their delivery. A liposomal system can transport H2 antagonists through environments in which they are normally degraded. Alternatively or additionally, a liposomal system can deliver H2 antagonists which are normally toxic in the free form. Alternatively or additionally, a liposomal system can release H2 antagonists slowly, over a prolonged period of time, thereby reducing the frequency of drug administration through an enhanced pharmacokinetic profile. Alternatively or additionally, a liposomal system can provide a method for forming an aqueous dispersion of hydrophobic H2 antagonists. Furthermore, liposomal systems may be directed to particular intracellular sites of interest.

Liposomes are substantially spherical structures made of materials having a high lipid content. The lipids in liposomes are organized in the form of lipid bilayers. Each bilayer is composed of two layers of lipids: an inner layer and an outer layer. The hydrophilic or polar ends of the lipids are oriented to form the external surface of the outer and inner layers. The hydrophobic or non-polar regions of the lipids self-assemble to form the interior of the bilayer.

Liposomes suitable for use in the practice of the present invention may be unilamellar vesicles (possessing a single membrane bilayer containing an entrapped aqueous volume), multilamellar vesicles (onion-like structures characterized by multiple membrane bilayers, each separated from the next by an aqueous layer), or paucilamellar vesicles (comprising about 2 to about 10 peripheral bilayers surrounding a large, unstructured (i.e., amorphous) central aqueous volume). In certain embodiments, the liposomes used in the compositions and methods of the present invention are paucilamellar vesicles.

Liposomes of the present invention may be from about 30 nm to about 2 microns in diameter, preferably about 50 nm to about 500 nm, more preferably about 60 nm to about 300 nm, and most preferably about 100 nm to about 300 nm.

Lipids and other Liposomes Components

Any of a number of lipids can be used to prepare liposomes for use in the present invention, including amphipathic, neutral, cationic, and anionic lipids. The term “amphipathic” has herein its art understood meaning and refers to a molecule having both hydrophobic and hydrophilic regions. A lipid for the formation of liposomes can be used alone or in combination with one or more other lipids.

Liposomes may be prepared using phospholipids such as phosphoglycerides and sphingolipids or non-phospholipids such as sphingolipids and glycosphingolipids. These lipids may also be mixed with other lipids including triglycerides and sterols (e.g., cholesterol). The selection of lipids for liposomes formation is generally guided by consideration of the needs with respect to the final liposomal size, the nature and characteristics of the H2 antagonist to be encapsulated, and the stability which is desired for the liposomal preparation.

Amphipathic lipids that can used for the formation of liposomes of the present invention include phospholipids, aminolipids, and sphingolipids. Non-limiting examples of phospholipids include shingomyelin, phosphatidylcholine, phosphatidylethanolamine, phosphatidylserine, phosphatidylinositol, phospatidic acid, palmitoyloleoyl, phosphatdylcholine, lysophosphatidylcholine, lysophosphatidyl-ethanolamine, dipalmitoylphosphatidylcholine, dioleolphosphatidylcholine, distearoylphosphatidylcholine, and dilinoleoylphosphatidylcholinie. Examples of amphipathic non-phospholipids include, but are not limited to, sphingolipids, glycosphingolipids, diacylglycerols, and β-acyloxyacids. Amphipathic lipids can be readily mixed with other lipids, such as triglycerides and sterols.

Cationic lipids, which carry a net positive charge at physiological pH, can readily be incorporated into liposomes. Such lipids include, but are no limited to N,N-dioleyl-N,N-dimethylammonium chloride, N-(2,3 -dioleyloxy)propyl-N,N,N-triethylammonium chloride, N,N-distearyl-N,N-dimethylammonium bromide, N-(2,3-dioleoyloxy)propyl)-N,N,N-trimethyl-ammonium chloride, 3β-(N-(N′,N′-dimethyl-aminoethane)-carbamoyl)cholesterol, N-(1-(2,3-dioleyloxy)propyl)-N-2-(spermine-carboxamido)ethyl)-N,N,-dimethylammonium trifluor-acetate, dioctadecylamino-glycyl carboxyspermine, 1,2-dileol-sn-3-phosphoethanolamine, and N-(1,2-dimyristyloxyprop-3-yl)-N,N-dimethyl-N-hydroxyethyl ammonium bromide. Preparation of cationic lipids are also available commercially including LIPOFECTIN and LIPOFECTAMINE (from GIBCO/BRL), and TRANSFECTAM (from Promego Corp.).

Anionic lipids (i.e., lipids which carry a net negative charge at physiological pH) suitable for use in the present invention include, but are not limited to, phosphatidylglycerol, cardiolipin, diacylphosphatidylcholine, diacylphosphatidic acid, N-dodecanoyl phosphatidyl-ethanolamine, N-succinyl phosphatidyl-ethanolamine, N-glutaryl phosphatidylethanolamine, and lysylphosphatidylglycerol. The inclusion of a negatively charged phospholipid may increase the stability of a liposomal preparation, preventing the spontaneous aggregation of the liposomes. In such preparation, the proportion of neutral phospholipid to anionic phospholipid may range from 5:1 to 1:1, respectively.

Neutral lipids (i.e., lipids which exist either in an uncharged or neutral zwitterionic form at physiological pH) include, but are not limited to, diacylphosphatidylcholine, diacylphosphotidylethanolamine, sphingomyelin, cephalin, sterols (e.g., cholesterol, see U.S. Pat. No. 4,721,612, which is incorporated herein by reference in its entirety), and tocopherols (e.g., cc-tocopherol, see U.S. Pat. No. 5,041,278, which is incorporated herein by reference in its entirety), and diacyglycerols. Inclusion of cholesterol in liposomes generally increases the stability of liposomes by decreasing the permeability of the membrane to ions and small polar molecules. Typically, the proportion of cholesterol to total lipids in the liposomes can vary from 0 to 50% (mol %).

Liposome can also include bilayer stabilizing agents such as polyamide oligomers (e.g., those described in U.S. Pat. No. 6,320,017, which is incorporated herein by reference in its entirety), peptides, proteins, and detergents. Liposomes may also be prepared to provide programmable fusion lipid formulations. Such formulations only fuse with cell membranes and deliver the encapsulated drug (e.g., a H2 antagonist) if a given signal event occurs. The signal event may be, for example, a change in pH, temperature, ionic environment, or time. In the latter case, clocking agents can be used, such as polyethylene glycol (PEG)-lipid conjugates (U.S. Pat. Nos. 5,820,873 and 5,885,613, each of which is incorporated herein by reference in its entirety). Such fusogenic liposomes are advantageous because the rate at which they become fusogenic can be not only pre-determined, but also varied as required over a time scale ranging from minutes to days. With other signal events, it is desirable to choose a signal that is associated with the disease site or target cell, such as increased temperature at a site of inflammation.

### Liposome Preparation

Liposomes for use in the compositions and methods of the present invention may be prepared using any of a variety of methods. Such methods are well-known in the art (see, for example, “Liposome Technology”, G. Gredoriadis (Ed.), 1991, CRC Press: Boca Raton, Fla.; D. Deamer and A. D. Bangham, Biochim. Biophys. Acta, 1976, 443: 629-634; Fraley et al., Proc. Natl. Acad. Sci. USA, 1979, 76: 3348-3352; F. Szoka et al., Ann. Rev. Biophys. Bioeng., 1980, 9: 467-508; Hope et al., Biochim. Biophys. Acta, 1985, 812: 55-65; L. D. Mayer et al., Biochim. Biophys. Acta, 1986, 858: 161-168; Hope et al., Chem. Phys. Lip., 1986, 40: 89-1-7; K. J. Williams et al., Proc. Natl. Acad. Sci., 1988, 85: 242-246; and U.S. Pat. Nos., 4,217,344; 4,235,871; 4,241,046; 4,356,167; 4,485,054; 4,551,288; 4,663,161; 4,737,323; 4,752,425; 4,774,085; 4,781,871; 4,877,561; 4,927,637;4,946,787; 5,190,822; 5,206,027; 5,498,420; 5,556,580 and 5,700,482, each of which is incorporated herein by reference in its entirety).

Suitable methods include, but are not limited to, sonication, extrusion, high pressure/homogenization, microfluidization, detergent dialysis, calcium-induced fusion of small liposomes vesicles and ether-infusion methods.

Liposomes may be prepared using the traditional thin-film method. In this method, the bilayer-forming elements (e.g., phosphatidylcholine, cholesterol, and, optionally, one or more anionic lipids) are mixed with a volatile organic solvent or solvent mixture (e.g., chloroform, ether, methanol, ethanol, butanol, cyclohexane, and the like). The solvent is then evaporated (e.g., using a rotary evaporator, a stream of dry nitrogen or argon, or other means) resulting in the formation of a dry lipid film. The film is then hydrated with an aqueous medium containing dissolved solutes, including buffers, salts, and hydrophilic compounds that are to be entrapped in the lipid vesicles. The hydration steps used influence the type of liposomes formed (e.g., the number of bilayers, vesicle size, and entrapment volume). The hydrated lipid thin film detaches during agitation and self-closes to form large, multilamellar vesicles (LMV) of heterogeneous sizes. The size distribution of the resulting multilamellar vesicles can be shifted toward smaller sizes by hydrating the lipids under more vigorous agitation conditions or by adding solubilizing detergents, such as deoxycholate. Alternatively or additionally, the vesicle size can be reduced by sonication or extrusion (see below).

Selection of suitable aqueous solutions for hydration of lipid thin-films is within the skill in the art. For example, the aqueous solution may be a buffered solution (pH range from 2.0 to 7.4) of the acid, sodium or ammonium forms of citrate or sulfate. Other suitable anionic acid buffers include phosphoric acid. Preferably, the temperature of the hydrating medium, before addition to the dry lipid film, is above the gel-liquid crystal transition temperature (Tc or Tm) of the lipid with the highest Tc. After addition of the hydrating medium, the lipid suspension is preferably maintained above the Tc during the hydration period. If desired, after formation of the vesicles, any un-entrapped buffers, salts or other hydrophilic compounds can be removed from the liposome dispersion, for example, by buffer exchange to 9% sucrose using either dialysis, size exclusion column chromatography (e.g., using Sephadex G-50 resin) or ultrafiltration (e.g., with 100,000-300,000 molecular weight cut-off).

Large unilamellar vesicles (LUVs) can be prepared using any of a variety of methods. For example, extrusion of MLVs through filters can provide LUVs whose sizes depend on the filter pore size used. In such methods (described, for example, in U.S. Pat. No. 5,008,050, which is incorporated herein by reference in its entirety), the MLV liposome suspension is repeatedly passed through the extrusion device resulting in a population of LUVs of homogeneous size distribution. The filtering may be performed through a straight-through membrane filter (e.g., Nucleopore polycarbonate filter of 30, 50, 60, 100, 200, or 800 nm pore size) or through a tortuous path filter (e.g., a Nucleopore membranfil filter of 0.1 μm size). When lipids having a gel to liquid crystal transition above ambient temperature are employed, an extruder having a heated barrel (or thermojacket) may be employed. LUVs may be exposed to at least one freeze-and-thaw cycle prior to the extrusion procedure as described by Mayer et al. (Biochim. Biophys. Acta, 1985, 817: 193-196).

Other methods for the preparation of unilamellar vesicles rely on the application of a shearing force to an aqueous dispersion of liposomes. Such methods include sonication and homogenization. Sonicating a liposome suspension using either a bath or probe sonicator leads to a progressive size reduction down to small unilamellar vesicles less than 50 nm in size. The size of the liposomal vesicles can be determined by quasi-elastic light scattering (QELs) (V. A. Bloomfield, Ann. Rev. Biophys. Bioeng., 1981, 10: 421-450). In a typical homogenization procedure, multilamellar vesicles are repeatedly circulated through a standard emulsion homogenizer at a pressure of 3,000 to 14,000 psi, preferably 10,000 to 14,000 psi and at a temperature corresponding to the gel-liquid crystal transition temperature of the lipid with the highest Tc, until selected liposome sizes, typically between about 100 and 500 nm, are observed.

Other techniques for preparing LUVs include reverse phase evaporation (U.S. Pat. No. 4,235,871, which is incorporated herein by reference in its entirety) and infusion procedures, and detergent dilution. For example, unilamellar vesicles can be produced by dissolving phospholipids in chloroform or ethanol and then injecting the lipids into a buffer, causing the lipids to spontaneously aggregate and form unilamellar vesicles. Alternatively, phospholipids can be solubilized into a detergent (e.g., cholates, Triton-X, or n-alkylglucosides). After formation of the solubilized lipid-detergent micelles, the detergent is removed by dialysis, gel filtration, affinity chromatography, centrifugation, ultrafiltration or any other suitable method.

Other liposomes that can be used in the present invention include those characterized as having substantially equal lamellar/solute distribution. These liposomes are known as stable plurilamellar vesicles or SPLV (such as those described in U.S. Pat. No. 4,522,803, which is incorporated herein by reference in its entirety) and include monophasic lipid vesicles or MPVs (such as those described in U.S. Pat. No. 4,588,578, which is incorporated herein by reference in its entirety).

### Paucilamellar Liposomes

In certain embodiments, liposomes used in the compositions and methods of the present invention are paucilamellar liposomes. Paucilamellar liposomes are composed of about 2 to about 10 concentric lipid bilayers surrounding a large, unstructured central cavity. Because of the dimensions of the central cavity, paucilamellar liposomes are particularly suited for transport of large quantities of aqueous-based materials. In addition, the multiple lipid bilayers of paucilamellar vesicles allow for transport of greater amounts of lipophilic materials and provide additional physical strength and resistance to degradation as compared with the single lipid bilayer of the large unilamellar vesicles. Furthermore, as shown in U.S. Pat. No. 4,911,928 (which is incorporated herein by reference in its entirety), the aqueous cavity of paucilemallar liposomes can be filled wholly or in part with an apolar oil or wax and can then be used as a vehicle for the transport or storage of hydrophobic materials. The amount of hydrophobic material which can be transported by paucilamellar vesicles with an apolar core is much greater than can be transported by the bilayers of multilamellar lipid vesicles.

Typically, paucilamellar liposomes useful in the compositions and methods of the present invention have approximately 2 to 8 lipid bilayers and range in diameter from about 100 to about 500 nm. Paucilamellar liposomes can comprise a variety of phospholipids, non-phospholipids, and single-tailed amphipathic molecules such as surfactants (R. A. Callow and J. J. McGrath, Cryobiology, 1985, 22: 251-267; D. F. H. Wallach and J. R. Philippot, in “Liposome Technology”, G. Gredoriadis (Ed.), 1991, CRC Press: Boca Raton, Fla., pp. 141-156; U.S. Pat. No. 5,147,723, each of which is incorporated herein by reference in its entirety).

Paucilamellar liposomes and methods for their preparation have been described in detail, for example in U.S. Pat. Nos. 4,911,928; 5,032,457; 5,104,736; 5,147,723; 5,160,669; 5,234,767; 5,251,425; 5,256,422; 5,474,848; 5,628,936; and 5,665,380 (each of which is incorporated herein by reference in its entirety).

In certain embodiments, paucilamellar liposomes for use in the compositions and methods of the present invention comprise surfactants. The terms “surfactant” and “surface active agent” are used herein interchangeably. They refer to a molecule that lowers the surface tension between two liquids. Typically, a surfactant is a linear organic molecule containing a hydrophobic group at one end and a hydrophilic group at the other end. Suitable surfactants for use in the preparation of paucilamellar vesicles include, but are not limited to, polyoxyethylene fatty acid esters and ether of various formulae; diethanolamines; long chain hexosamides; acyl amino acid amides and acylamides of various formulae; polyoxyethylene glyceryl monostearates and glycerol monostearate, glycerol palmitate, and glycerol oleate. Surfactants including the BRIG family of polyoxyethylene acyl ethers, the SPAN sorbitan alkyl esters, and the TWEEN polyoxyethylene sorbitan fatty acid esters are commercially available, for example, from ICI, Inc. (London, UK).

The incorporation of a charge-producing component, yielding a net positive or net negative charge to the lipid paucilamellar vesicles may be desirable. As already mentioned above, the incorporation of a charge-bearing material has been reported to stabilize the lipid structure of liposomes. Non-limiting examples of suitable negative charge-producing molecules include oleic acid, dicetyl phosphate, palmitic acid, cetyl sulphate, retinoic acid, phosphatidic acid, phosphatidyl serine, and combinations thereof. Examples of suitable positive charge-producing molecules include, but are not limited to, stearyl amines or oleyl amines, cetyl pyridinium chloride, quaternary ammonium compounds (e.g., Quaternium-14, Quaternium-18, Quaternium-18 methosulfate, and cetyl trimethyl ammonium bromide, chloride or tosylate), or combinations thereof

Alternatively or additionally, a sterol, such as a cholesterol or one of its derivatives, or a sterol-like molecule may be incorporated in paucilamellar vesicles. Incorporation of sterols in the lipid bilayers of paucilamellar liposomes has been found to increase the stability of the bilayer and to provide optimum size control of the finished liposome.

In certain embodiments, the paucilamellar vesicles are partly or substantially filled with a water immiscible oily solution. Examples of suitable water immiscible oily solutions include, but are not limited to, mineral oil, silicone oils such as dimethicone, cyclomethicone, and the like, natural and synthetic triglycerides, acyl esters, and petroleum derivatives, and their analogues and derivatives. Methods for preparing such paucilamellar vesicles have been described, for example, in U.S. Pat. No. 4,911,928 (which is incorporation herein by reference in its entirety).

Alternatively or additionally, paucilamellar vesicles suitable for use in the practice of the present invention include targeting molecules, which can be used to direct the vesicles to a particular target in order to allow release of the material encapsulated in the vesicle at a specified biological location (see, for example, U.S. Pat. No. 5,665,380, which is incorporated herein by reference in its entirety). Examples of targeting molecules include, but are not limited to, monoclonal antibodies, other immunoglobulins, lectins, peptide hormones, and neutral or charged glycolipids. Such targeting molecules may be covalently attached to components of the lipid bilayers (e.g., surfactant molecules) either directly or indirectly (e.g., through a linker). Alternatively or additionally, the targeting molecules may interact (e.g., through hydrogen bonds) with components of the lipid bilayers.

In certain embodiments, the paucilamellar liposomes used in the compositions and methods of the present invention are liposomes commercially available from Micro Vesicular Systems, Inc (Nashua, N.H.), a subsidiary of ICI, Inc (London, UK), under the trademark NOVASOME®. These paucilamellar liposomes have been described in detail in U.S. Pat. No. 5,628,936 (which is incorporated herein by reference in its entirety).

### Liposome Encapsulation of H2 Antagonists

Any of a number of methods can be used to load one or more H2 antagonists into liposomes. As will be appreciated by one of ordinary skill in the art, a H2 antagonist may be associated with the lipid bilayer(s) of a liposome or incorporated into the interior of the liposome, or both. Accordingly, the terms “entrapped” and “encapsulated” are taken herein to include both the drug inside the internal cavity as well as the drug associated with the lipid bilayer(s) of the liposomes.

In certain embodiments, the method used for loading liposomes with H2 antagonists exhibits a loading efficiency (i.e., provides a percent encapsulated H2 antagonist) of 50% or greater, 60% or greater, 75% or greater, or 90% or greater. Any lipid:H2 antagonist molar ratio that is sufficient for the liposomal preparation to fulfill its intended purpose (e.g., prevention of periodontal disease) is contemplated by the present invention. In certain embodiments, the lipid:H2 antagonist molar ratio is between 5:1 and 100:1, or between 10:1 and 40:1 or between 15:1 and 25:1.

Loading of liposomes with H2 antagonists may be performed by any suitable method. The simplest method of loading is a passive entrapment of a water soluble material (e.g., water soluble H2 antagonist) in the dry lipid film mentioned above by hydration of lipid components in a process similar to that described above. The H2 antagonist and liposome components may be dissolved in an organic solvent and concentrated to a dry film. A buffer is then added to the dried film and liposomes are formed having the H2 antagonist incorporated into the vesicles. Alternatively, the H2 antagonist can be placed into a buffer and added to a dried lipid film.

Other loading techniques include the dehydration-rehydration method in which pre-formed liposomes are dehydrated in the presence of a H2 antagonist and subsequently reconstituted. Alternatively, a H2 antagonist compound that is not water soluble at room temperature can be passively loaded by incubating the compound with pre-formed liposomes at a temperature at which the H2 antagonist is relatively water soluble, allowing the compound to equilibrate with and diffuse into the liposomes, and then lowering the temperature, which leads to precipitation of the compound within the liposomes.

Other methods of passively loading pre-formed liposomes include transmembrane permeation using alcohols, such as ethanol, as described, for example in U.S. Pat. No. 6,447,800 (which is incorporated herein by reference in its entirety). Such methods comprise combining a dispersion of liposomes and an alcohol which increases the membrane permeability of the liposomes to the solute (e.g., H2 antagonist). The alcohol temporarily enhances the permeability of the vesicles, without substantially altering or changing their size, so that solutes added to the extra-liposomal space equilibrate with the internal space. Subsequent dilution of the alcohol returns the permeability barrier to its normal level, thus trapping the solute inside the liposome.

Other suitable loading methods include active transmembrane loading techniques, in which conditions are provided under which the substance to be encapsulated can penetrate into the pre-formed liposome through its walls. A transmembrane chemical potential may be employed to drive the substance to be loaded into the liposome. Typically, the transmembrane potential is created by a concentration gradient which is formed by having different concentrations of a particular species on either side of the liposomal membrane. Neutralization of the concentration gradient is associated with the substance being loaded into the liposome. pH gradients (U.S. Pat. Nos. 4,946,683; 5,192,549; 5,204,112; 5,262, 168; and 5,380,531, which are all incorporated herein by reference in their entireties), Na⁺/K⁺ gradients (U.S. Pat. Nos. 5,171,578; and 5,077,056, each of which is incorporated herein by reference in its entirety), and NH₄⁺ gradients (U.S. Pat. No. 5,316,771, which is incorporated herein by reference in its entirety) have been used to load a variety of drugs into liposomes. Such methods can be applied for loading H2 antagonists that are ionizable or protonable into liposomes.

Other chemical potential driven methods for liposome loading after liposome formation have used a concentration gradient of the solute itself to drive the loading process by employing liposomes with low ionic strength interiors and raising the temperature above the gel-liquid crystal transition temperature or temporarily disrupting the liposome membrane with shear stresses (as described, for example, in U.S. Pat. No. 5,393,350; 5,104,661 and 5,284,5881, each of which is incorporated herein by reference in its entirety).

After loading, any un-entrapped H2 antagonist may be removed from the liposome dispersion, for example by buffer exchange to 9% sucrose using either dialysis, size exclusion column chromatography (e.g., using a Sephadex G-50 resin) or ultra-filtration (using a 100,000 or 300,000 molecular weight cut-off).

### Liposomal Dehydration and Storage

If desired, liposomes for use in the compositions and methods of the present invention may be lyophilized or dehydrated at various stages of formation. For example, the lipid film may be lyophilized after removal of the solvent and prior to addition of the H2 antagonist. Alternatively, the lipid-H2 antagonist thin-film may be lyophilized prior to hydration and formation of liposomes. Alternatively, “empty” liposomes may be lyophilized before encapsulation of the H2 antagonist; or loaded liposomes may be lyophilized before being used.

Dehydration may be carried out using any suitable method, including by exposing the lipids or liposomes to reduced pressure without prior freezing (as described, for example, in U.S. Pat. Nos. 4,229,360 and 4,880,635, each of which is incorporated herein by reference in its entirety) or with prior freezing (as described, for example, in U.S. Pat. No. 4,311,712, which is incorporated herein by reference in its entirety). Freezing may be performed by placing the lipid or liposome preparation in surrounding medium in liquid nitrogen.

Liposomal dehydration is typically performed in the presence of a hydrophilic drying protectant (U.S. Pat. No. 4,880,635, which is incorporated herein by reference in its entirety). This hydrophilic drying agent is generally believed to prevent the rearrangement of the lipids in the liposome, so that the size and contents are maintained during the drying procedure and through rehydration, such that the liposomes can be reconstituted. Suitable drying protectants include any molecule that can form strong hydrogen bonds, and that possesses stereochemical features that preserve the intramolecular spacing of the liposome bilayer components. Saccharide sugars, in particular mono- and disaccharides, are suitable for use as drying protectants for liposomes. Generally, the protective sugar concentration in the lipid or liposome preparation prior to dehydration is from about 100 mM to about 250 mM.

After dehydration, the lipids or liposomes can be stored for extended periods of time until they are to be used. Selecting the appropriate temperature for storage is within the skill in the art and will depend on the lipid formulation of the liposomes and the temperature sensitivity of encapsulated materials.

### III. Uses of Liposome-Encapsulated H2 Antagonists

In another aspect, the present invention provides methods for the improved local delivery of H2 antagonists in a subject (e.g., human or other mammal). The inventive methods generally comprise topically administering, to a subject in need thereof, a liposome-encapsulated H2 antagonist as described above.

The inventive methods of improved local delivery of H2 antagonists may be used for the prevention and/or treatment of any disease state or condition for which topical administration of a H2 antagonist is beneficial.

Such disease states or conditions include, for example, periodontal diseases. As reported in the Examples below, the present Applicants have shown that topical administration of a liposomal preparation of a H2 antagonist prevents gingival inflammation and bone destruction in a rabbit periodontitis model. More specifically, topical delivery of a NOVASOME® (i.e., paucilamellar vesicles) preparation of the H2 antagonist Cimetidine was found to prevent bone loss, inflammatory cell infiltration, and connective tissue destruction that is otherwise observed in P. gingivalis-induced periodontitis in rabbits.

Accordingly, the present invention provides methods for preventing or treating periodontal disease in a subject. The inventive methods comprise topically administering to the subject an effective amount of a liposome-encapsulated H2 antagonist.

The term “periodontal diseases” include all diseases of the periodontal tissues that surround and support the teeth (see, for example, D. M. Williams et al., “Pathology of Periodontal Disease”, 1992, Oxford University Press). These include the gingiva, cementum, periodontal ligament, alveolar process bone, and dental supporting bone. Specifically, periodontal diseases include gingivitis and periodontitis. Gingivitis is a disease in which inflammation is localized within the gingiva and no lesion occurs in the bone between the teeth and gingiva. Periodontitis is a disease in which gingival inflammation reaches the periodontal ligament and alveolar bone. Left untreated, periodontitis can lead to tooth loss.

It is to be understood that compositions and methods of the present invention may also be used to prevent or treat secondary diseases that are related to a periodontal disease. As already mentioned above, periodontal disease is known to have implications beyond the deleterious effects on oral tissues and structural integrity. In particular, periodontal disease represents a potential risk factor for increased morbidity or mortality in pregnancy and for several other systemic diseases including cardiovascular disease and diabetes (R. C. Page et al., Ann. Periodontol., 1998, 3: 108-120; R. I. Garcia et al., Ann. Periodontol., 1998, 3: 339-349). In this context, it has been shown that local infection with the periodontal pathogen P. gingivalis up-regulates the expression of COX-2, a marker of on-going inflammation, in lung associated tissues (U.S. Pat. Appln. No. 2004/0019110 by Van Dyke et al.). In view of these results, the prevention or treatment of periodontal disease is likely to have a beneficial impact on the prognosis of a number of systemic diseases. Thus, the present invention is also related to methods for treating systemic diseases that are related to periodontal disease, such as cardiovascular diseases, pregnancy complications, and diabetes. These methods also comprise topically administering to a subject an effective amount of a liposome-encapsulated H2 antagonist.

The compositions and methods of the present invention may be used to prevent or treat diseases of other oral tissues, e.g., without limitation, aphthous ulcers and herpetic stomatitis. Aphthous ulcers can be classified into three different types: minor, major and herpetiform (J. A. Burgess et al., Drugs, 1990, 39: 54-65; I. M. Freedberg in “Fitzpatrick's Dermatology in General Medicine”, 5th Ed., Vol. 1, 1999, McGraw-Hill: New York, N.Y.). Minor aphthae are generally located on labia or buccal mucosa, the soft palate and the floor of the mouth. They can be singular or multiple, and tend to be small (less than 1 cm in diameter) and shallow. Major aphthae are larger and involve deeper ulceration. Major aphthae may also be more likely to scar with healing. Herpetiform aphthae frequently are more numerous and vesicular in morphology. Herpetic stomatitis is a viral infection of the mouth caused by Herpes simplex virus (or HSV) and characterized by ulcers and inflammation. Herpetic stomatitis is often seen in young children. This condition probably represents their first exposure to herpes virus and can result in a systemic illness characterized by high fever, blisters, ulcers in the mouth, and inflammation of the gums.

Compositions and methods of the present invention may also find applications in the treatment and/or prevention of inflammatory skin disorders. Thus, liposome-encapsulated H2 antagonists, alone or in combination with one or more other therapeutic agents, may be topically administered for preventing or treating various skin and/or mucosal conditions including, but not limited to, psoriasis, atopic eczema, urticaria, allergic reactions, and warts. For example, topical application of Cimetidine to the oral cavity of an HIV-positive adult with recalcitrant mucosal warts was shown to induce complete resolution of intra- and peri-oral warts (0. Wargon, Austral. J. Dermatol., 1996, 37: 149-150). Topical administration of Cimetidine and Cetirizine, a H1 antagonist, has been found to be useful to control burn wound itch (R. A. U. Baker et al., J. Burn Care & Rehabilitation, 2001, 22: 263-268). The availability of improved systems for the local delivery of H2 antagonists provided herein is likely to increase the use of H2 antagonists for the treatment or prevention of skin/mucosa conditions.

### IV. Pharmaceutical Compositions

An inventive liposomal preparation of H2 antagonists can be administered per se or as a pharmaceutical composition. Accordingly, the present invention provides pharmaceutical compositions comprising a liposome-encapsulated H2 antagonist admixed with at least one physiologically acceptable carrier or excipient.

Depending on the mode of administration, the inventive pharmaceutical compositions may be in the form of liquid, solid, or semi-solid dosage preparation.

For example, the compositions may be formulated as solutions, dispersion, suspensions, emulsions, mixtures, lotions, liniments, jellies, ointments, creams, pastes including toothpastes, gels, hydrogels, aerosols, sprays including mouth sprays, powders including tooth powders, granules, granulates, lozenges, salve, chewing gum, pastilles, sachets, mouthwashes, tablets, dental floss, plasters, bandages, sheets, foams, films, sponges, dressings, drenches, bioadsorbable patches, sticks, and the like.

### Formulation

The pharmaceutical compositions of the present invention may be formulated according to general pharmaceutical practice (see, for example, “Remington's Pharmaceutical Sciences” and “Encyclopedia of Pharmaceutical Technology”, J. Swarbrick and J. C. Boylam (Eds.), 1988, Marcel Dekker, Inc.: New York, N.Y.). Preferably, the pharmaceutical compositions are formulated for topical administration.

Physiologically acceptable carriers or excipients for use with the inventive pharmaceutical compositions can be routinely selected for a particular use by those skilled in the art. These include, but are not limited to, solvents, buffering agents, inert diluents or fillers, suspending agents, dispersing or wetting agents, preservatives, stabilizers, chelating agents, emulsifying agents, anti-foaming agents, gel-forming agents, ointment bases, penetration enhancers, humectants, emollients, and skin protecting agents.

Examples of solvents are water, alcohols, vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, and diethylamine Suitable suspending agents are, for example, naturally occurring gums (e.g., acacia, arabic, xanthan, and tragacanth gum), celluloses (e.g., carboxymethyl-, hydroxyethyl-, hydroxypropyl-, and hydroxypropylmethyl-cellulose), alginates and chitosans. Examples of dispersing or wetting agents are naturally occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate).

Preservatives may be added to a pharmaceutical composition of the invention to prevent microbial contamination that can affect the stability of the formulation and cause infection in the patient. Suitable examples of preservatives include parabens (such as methyl, ethyl, propyl, p-hydroxybenzoate, butyl, isobutyl, and isopropylparaben), potassium sorbate, sorbic acid, benzoic acid, methyl benzoate, phenoxyethanol, bronopol, bronidox, MDM hydantoin, iodopropynyl butylcarbamate, benzalconium chloride, cetrimide, and benzylalcohol. Examples of chelating agents include sodium EDTA and citric acid.

Examples of emulsifying agents are naturally occurring gums, naturally occurring phosphatides (e.g., soybean lecithin; sorbitan mono-oleate derivatives), sorbitan esters, monoglycerides, fatty alcohols, and fatty acid esters (e.g., triglycerides of fatty acids). Anti-foaming agents usually facilitate manufacture, they dissipate foam by destabilizing the air-liquid interface and allow liquid to drain away from air pockets. Examples of anti-foaming agents include simethicone, dimethicone, ethanol, and ether.

Examples of gel bases or viscosity-increasing agents are liquid paraffin, polyethylene, fatty oils, colloidal silica or aluminum, glycerol, propylene glycol, carboxyvinyl polymers, magnesium-aluminum silicates, hydrophilic polymers (such as, for example, starch or cellulose derivatives), water-swellable hydrocolloids, carragenans, hyaluronates, and alginates. Ointment bases suitable for use in the compositions of the present invention may be hydrophobic or hydrophilic, and include paraffin, lanolin, liquid polyalkylsiloxanes, cetanol, cetyl palmitate, vegetable oils, sorbitan esters of fatty acids, polyethylene glycols, and condensation products between sorbitan esters of fatty acids, ethylene oxide (e.g., polyoxyethylene sorbitan monooleate), and polysorbates.

Examples of humectants are ethanol, isopropanol glycerin, propylene glycol, sorbitol, lactic acid, and urea. Suitable emollients include cholesterol and glycerol. Examples of skin protectants include vitamin E, allatoin, glycerin, zinc oxide, vitamins, and sunscreen agents.

The pharmaceutical compositions of the invention may, alternatively or additionally, comprise other types of excipients including, thickening agents, bioadhesive polymers, and permeation enhancing agents.

Thickening agents are generally used to increase viscosity and improve bioadhesive properties of pharmaceutical compositions. Examples of thickening agents include, but are not limited to, celluloses, polyethylene glycol, polyethylene oxide, naturally occurring gums, gelatin, karaya, pectin, alginic acid, and povidone. Particularly interesting are thickening agents with thixotropic properties (i.e., agents whose viscosity is decreased by shaking or stirring). The presence of such an agent in a pharmaceutical composition allows the viscosity of the composition to be reduced at the time of administration to facilitate its application to the site of interest (e.g., to the gingiva or periodontal pocket) and, to increase after application so that the composition remains at the site of administration.

In embodiments where an inventive pharmaceutical composition is intended to be applied on skin, bioadhesive polymers are useful to hydrate the skin and enhance its permeability. Bioadhesive polymers can also function as thickening agents. Examples of bioadhesive polymers include, but are not limited to, pectin, alginic acid, chitosan, polysorbates, poly(ethyleneglycol), oligosaccharides and polysaccharides, cellulose esters and cellulose ethers, and modified cellulose polymers. Permeation enhancing agents are vehicles containing specific agents that affect the delivery of active components through the skin. Permeation enhancing agents include solvents, such as alcohols (e.g., ethyl alcohol, isopropyl alcohol), dimethyl formamide, dimethyl sulfoxide, 1-dodecylazocyloheptan-2-one, N-decyl-methylsulfoxide, lactic acid, N,N-diethyl-m-toluamide, N-methyl pyrrolidone, nonane, oleic acid, petrolatum, polyethylene glycol, propylene glycol, salicylic acid, urea, terpenes, and trichloroethanol) and surface active compounds.

In embodiments where an inventive pharmaceutical composition is intended to be applied on skin, the pharmaceutical composition may be packaged as kits comprising a container including the liposome-encapsulated H2 antagonist, optionally admixed with physiologically acceptable carriers or excipients, and at least one dressing, wherein the dressing is to be applied to cover the skin site following local administration of the content of the container to the site. The term “dressing” refers to any covering designed to protect a skin site. The term includes porous and non-porous coverings, woven and non-woven coverings, absorbent coverings, and occlusive coverings. The dressing may also be used as a delivery system for the pharmaceutical composition. For example, the pharmaceutical composition may be incorporated into or coated onto the dressing (e.g., by dipping the dressing in or spraying the dressing with the liposomal suspension).

In embodiments where an inventive pharmaceutical composition is intended to be administered to the oral cavity, the composition may desirably comprise other components, such as, for example, topical oral carriers. Such carriers include, but are not limited to, anticaries agents, antiplaque agents, anticalculus agents, anti-inflammatory agents, dental abrasives, flavoring agents, sweetening agents, binders, humectants, thickening agents, buffering agents, preservatives, coloring agents, and pigments, flavorants, fillers, stabilizers, ethanol and water.

### Dosage

Generally, a pharmaceutical composition according to the present invention, comprises an effective amount of a liposome-encapsulated H2 antagonist. The effective amount may be a “prophylactically effective amount” or a “therapeutically effective amount”. The term “prophylactically effective amount” refers to an amount effective at dosages and for periods of time necessary to achieve the desired prophylactic result (e.g., prevention of periodontal disease). Typically, since a prophylactic dose is used in a subject prior to or at an early stage of disease, the prophylactically effective amount will be lower than the therapeutically effective amount. The term “therapeutically effective amount” refers to an amount effective at dosages and for periods of time necessary to achieve the desired therapeutic result (e.g., a decrease in the extent or severity of symptoms of the disease). A therapeutically effective amount of a liposome-encapsulated H2 antagonist may vary according to factors such as the disease state, age, sex and weight of the subject, and the ability of the liposome-encapsulated H2 antagonist to elicit a desired response in the subject.

A prophylactically or therapeutically effective amount is also one in which any toxic or detrimental effects of the H2 antagonist are outweighed by the beneficial effects.

The prophylactically effective amount and/or therapeutically effective amount can be estimated initially either in cell culture assays or in animal models, usually mice, rabbits, dogs or pigs. The animal model may also be used to achieve a desirable concentration range and route of administration. Such information can then be used to determine useful doses and routes of administration in other subjects (e.g., human patients).

The total dose required for each treatment may be administered by multiple doses or in a single dose. Adjusting the dose to achieve maximal efficacy based on these or other methods are well known in the art and are within the capabilities of trained physicians.

### Administration

The mode of administration of a pharmaceutical composition of the invention will mainly depend on the form of the preparation chosen. For example, gels, lotions, creams and ointments may be manually applied or sprayed (either with a manually-activated pump or with the aid of a suitable pharmaceutically acceptable propellant) onto the surface area in need of treatment. Alternatively, a brush, syringe, spatula or a specifically designed container (such as tube with a narrow tip) can be used to apply an inventive pharmaceutical composition. For administration in the oral cavity, mouthwashes, toothpastes, mouth sprays, chewing gums, dental floss may also be useful.

### Other Therapeutic Agents

In certain embodiments, the liposome-encapsulated H2 antagonist is the only active ingredient in an inventive pharmaceutical composition. In other embodiments, the pharmaceutical composition further comprises one or more other therapeutic agents. In still other embodiments, the pharmaceutical composition further comprises a combination of therapeutic agents.

Therapeutic agents that can be included in the pharmaceutical compositions of the present invention include, but are not limited to, analgesics, anesthetics, antimicrobial agents, antibacterial agents, antiviral agents, antifungal agents, antibiotics, anti-inflammatory agents (e.g., non-steroid anti-inflammatory agents (NSAIDs) such as COX-2 inhibitors including celcoxib, rofecoxib, and/or valdecoxib), antioxidants, antiseptic agents, other antihistamine agents (e.g., H1 antagonists), antipruritic agents, antipyretic agents, immunostimulating agents, and dermatological agents.

It will be appreciated that, in the methods of the present invention, liposome-encapsulated H2 antagonists can be employed in combination therapies (i.e., the liposomal composition can be administered concurrently with, prior to, or subsequent to one or more desired therapies of medical procedures). The particular combination of therapies (therapeutics or procedures) to employ in a combination regimen will usually take into account compatibility of the desired therapeutics and/or procedures and the desired prophylactic or therapeutic effect to be achieved.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Some of the results presented in this section have recently been described by the Applicants in a scientific publication (H. Hasturk et al., “Topical H2-Antagonist Prevents Experimental Periodontitis in Rabbit Model”). This article is incorporated herein by reference in its entirety.

### Topical Administration of Cimetidine/NOVASOME® to Experimental Periodontitis

The purpose of this study was to analyze the histopathological changes associated with experimental periodontitis in response to topically applied cimetidine/NOVASOME®.

### A. Materials and Methods

### Animal Model

Study protocol and experimental design have been reviewed and approved by the Boston University Medical Center Institutional Animal Care and Use Committee (BUMC IACUC) prior to study initiation (IACUC protocol # AN-13948). In addition, BUMC Institutional Biohazard Committee (IBC) has approved the use of Porphyromonas gingivalis (P. gingivalis) in this animal model to induce periodontal disease (IBC protocol # A-269).

In total, 21 New Zealand White rabbits (male, 3.5-4.0 kg each) were used in the experiments presented herein. Three different doses of Cimetidine (0.1, 1.0 and 10 μg/mL) have been prepared in paucilamellar liposomes (NOVASOME®). The animals were distributed as follows: Group A: Ligature alone (2 rabbits); Group B: Ligature+P. gingivalis (4 rabbits); Group C: Ligature+P. gingivalis+liposome (NOVASOME®) alone; Group D: Ligature+P. gingivalis+NOVASOME® preparation comprising 0.1 μg/mL of Cimetidine (4 rabbits); Group E: Ligature+P. gingivalis+NOVASOME® preparation comprising 1.0 μg/mL of Cimetidine (4 rabbits); and Group F: Ligature+P. gingivalis+NOVASOME® preparation comprising 10 μg/mL of Cimetidine (4 rabbits). All animals were purchased from Pine Acre Farms (Berthoud, Co.). The weight of the animals was strictly controlled and all animals weighed between 3.5-4.0 kg at the time of the initial experimentation. The animals were kept in individual cages, received water ad libitum, and were fed with specialized food (chow) for at least 5 days for acclimatization by experienced and licensed laboratory technicians at the Laboratory Animal Science Center at BUMC (BUMC LASC).

### Experimental Periodontitis

Ligature placement was performed under general anesthesia using ketamine (40 mg/kg) and xylazine (5 mg/kg) injections. Animals had a 3-0 silk suture placed around the second pre-molar of both mandibular quadrants. Animals in group A received ligature only while animals in groups B, C, D, E, and F received P. gingivalis in addition to ligature placement.

P. gingivalis (strain A74376) was grown as previously described. Briefly, bacteria were cultured on agar plates containing trypticase soy agar supplemented with 0.5% (w/v) yeast extract, 5% defibrinated sheep red blood cells, 5 μg hemin, and 1 μg/mL vitamin K. Plates were incubated for 3 days at 37° C. in jars anaerobically maintained through palladium catalyzed hydrogen/carbon dioxide envelopes (GasPak Plus, BD Microbiology Systems, Sparks, Md.). Colonies were randomly selected and anaerobically cultured overnight at 37° C. in Schaedler's broth supplemented with vitamin K and hemin. Bacteria numbers were spectrophotometrically determined at 600 nm and 10⁹ CFU (0.8 OD) were mixed with carboxymethylcellulose to form a thick slurry, which was applied topically to the ligated teeth. The sutures were checked at every appointment, and lost or loose sutures were replaced.

### Topical Application of Cimetidine

Topical application was performed in animals of Groups D, E, and F every other day for 6 weeks under inhalation anesthesia using isoflurane (4.0 MAC/2.0 MAC). In these groups, Cimetidine was applied at three different doses. Cimetidine preparations were delivered in liposomes (NOVASOME®) that served as a vehicle for the H2 receptor antagonist. In order to see if liposome application alone would have any effect on the outcome, animals in Group C received liposomes without Cimetidine (i.e., NOVASOME® alone) in addition to ligature placement and P. gingivalis.

At the end of the study, the animals were euthanized using overdose pentobarbital (euthanasia) injections (120 mg/kg) according to the approved protocol by the IACUC. No adverse events were observed during experimental procedures throughout the study with regard to the animal care and no animals were prematurely lost during the study.

### Morphometric Analysis

After sacrificing the animals, the mandible was dissected free of muscles and soft tissue, keeping the attached gingiva intact with the bone. Then the mandible was split into two halves from the midline between the central incisors. One half was taken for morphometric analysis of bone loss and the other half was used for histological evaluation of periodontitis.

Half of the sectioned mandible was defleshed by immersing in 10% hydrogen peroxide (10 minutes, room temperature). The soft tissue was removed carefully and then the mandible was stained with methylene blue for good visual distinction between the tooth and the bone. Next, the bone level around the second pre-molar was measured directly by a 0.5 mm calibrated periodontal probe. Measurements were made at three points each, at buccal and lingual sides, for crestal bone level. A mean crestal bone level around the tooth was calculated. Similarly, for the proximal bone level, measurements were made at mesial and distal aspects of the tooth. The measurements were taken from both the buccal and lingual sides on both proximal aspects of the second premolar and the mean proximal bone level was calculated. The bone level was also quantified by Image Analysis (Image-Pro Plus 4.0, Media Cybernetics, Silver Spring, Md.). The sectioned mandible was mounted and photographed using an inverted microscope at 10×. The captured image was also analyzed as above and the mean crestal bone level around the tooth was calculated in millimeters.

### Radiographic Analysis

The percentage of the tooth within the bone was calculated radiographically using Bjorn technique (A. Jain et al., Infect. Immun., 2003, 71: 6012-6018). The radiographs were taken with a digital X-ray (Schick Technologies Inc., Long Island City, N.Y.). To quantify bone loss, the length of the tooth from the cusp tip to the apex of the root was measured, as was the length of the tooth structure outside the bone, measured from the cusp tip to the coronal extent of the proximal bone. From these measurements, the percentage of the tooth within the bone was calculated. Bone values are expressed as the percentage of the tooth in the bone (i.e., [length of tooth in bone×100]/total length of tooth).

### Histological Analysis

For histological analysis, the other half of the mandible was immersed in a volume of immunocal (Decal Corporation, Tallman, N.Y.) equal to at least 10 times the size of the section; the solution was replaced every 24 hours for 72 hours. Decalcification was assessed and confirmed by serial radiographs, which were taken every other day during two weeks. After decalcification, the tissues were rinsed for 1-3 minutes in running water, placed in Cal-Arrest (Decal Corporation, Tallman, NY) in order to neutralize the pH of the tissue, enhance embedding and staining characteristics, and stop further decalcification so that the tissue does not become over-decalcified.

The tissue was kept in this solution for 2-3 minutes, rinsed again in flowing deionized water for at least 3 minutes and kept in formalin for at least 24 hours before embedding in paraffin. Thin sections (5 μm) were cut and sections were conventionally stained with hematoxylin and eosin (H&E) to identify the cellular composition of the inflammatory infiltrates, and one hundred seventy (170) 5 μm-sections were stained with tartrate-resistant acid phosphatase (TRAP) to detect osteoclastic activity.

### Statistical Analysis

The data obtained by direct measurements during morphologic assessment and by histomorphometric measurements was used in multiple statistical analyses. Mean values for linear and area measurements were utilized to determined the changes in bone level during the topical application of P. gingivalis, liposome, and liposome+Cimetidine combinations. The ratio calculations were used and multiple comparisons within groups were made using analysis of variance (ANOVA) with Bonferroni correction.

### B. Results

### Macroscopic Analysis

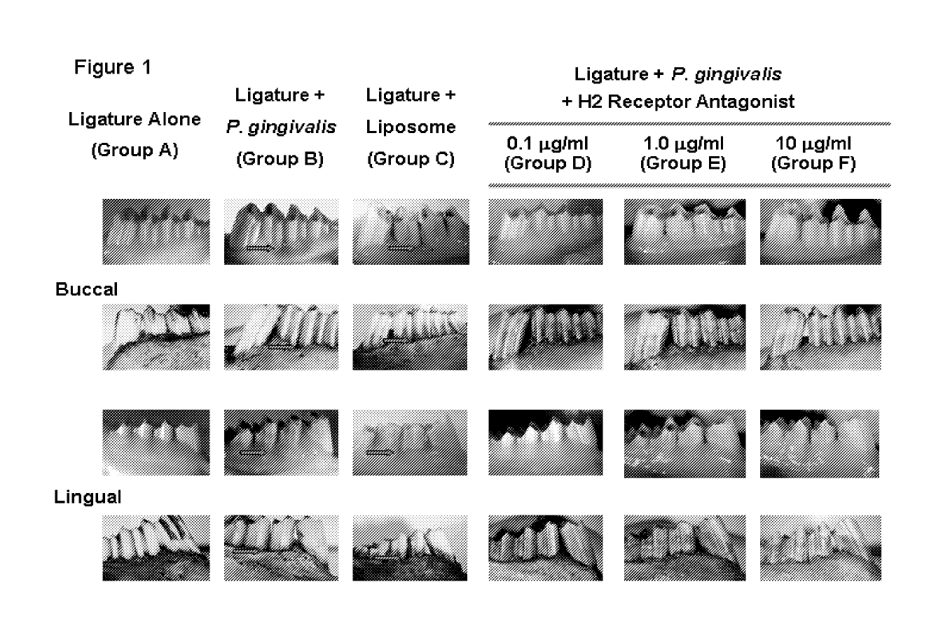

FIG. 1 shows the mandibles of rabbits treated either with ligature alone or ligature and topical P. gingivalis application and which then received either different doses of Cimetidine or the vehicle alone (liposome). This figure shows gingival tissue and defleshed bone specimens from buccal or lingual aspects. Ligature placement without additional P. gingivalis application did not lead to any significant soft or hard tissue changes in rabbit mandibles (animals of Group A). Arrows depict the soft and hard tissue changes in Groups B and C of animals. Topical delivery of three different doses of Cimetidine before P. gingivalis application prevented the gingival inflammation and bone destruction in a significant and comparable way with no apparent dose-dependent effect (Groups D, E, and F).

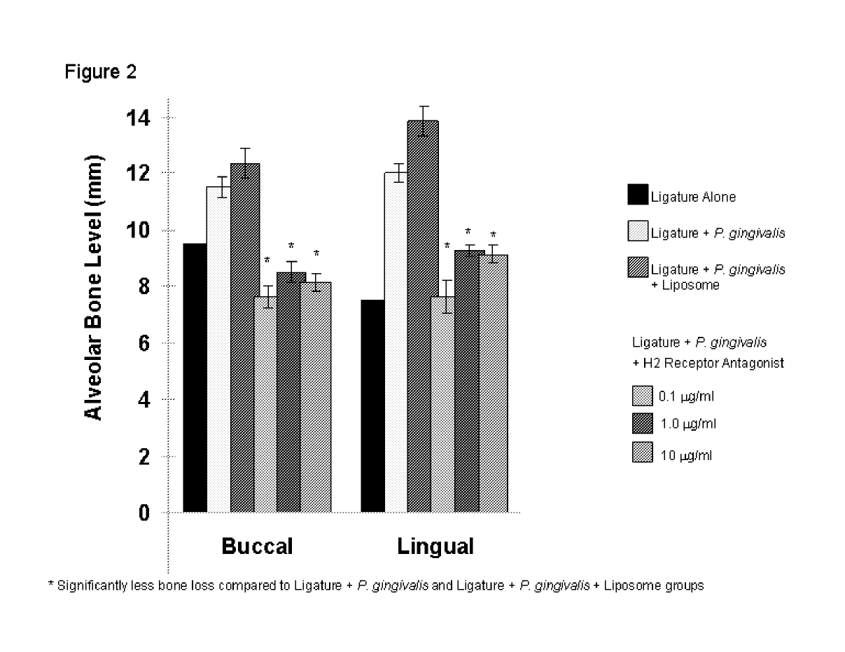

FIG. 2 shows the results of quantitative analyses of defleshed bone specimens. The findings demonstrate that preventive effects of Cimetidine on P. gingivalis and ligature-induced experimental periodontitis in rabbits are statistically significant compared to animals that have received liposome (NOVASOME®) as placebo where the bone loss was significantly higher (p<0.05, ANOVA). These preventive effects of Cimetidine were similar with all three doses used in this study.

### Radiographic Analysis

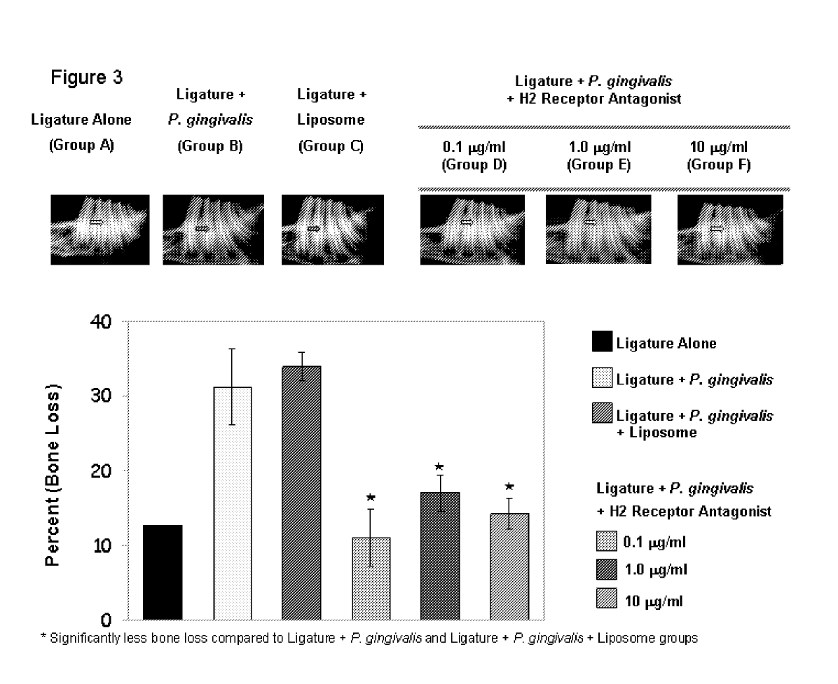

FIG. 3 shows the radiographic analyses of bone and other hard tissue components. The upper panel demonstrates the bone loss in animals that have received ligature placement+P. gingivalis, and in animals that have received ligature placement+P. gingivalis and vehicle (liposome) (Groups B and C). The bone loss (indicated by an arrow in B and C) is visible and significantly different compared to animals that have received ligature alone (Group A). Topical application of Cimetidine (all three doses) prevented bone loss and the radiographic appearance of alveolar bone revealed bone levels identical to those of animals that received the ligature application alone (see arrows, Groups D, E and F).

The lower panel of FIG. 3 presents the percentage of bone loss as calculated by Bjorn technique. This measurement further confirmed that Cimetidine application prevents the destructive effects of P. gingivalis-induced periodontitis (p<0.05 compared to Cimetidine or ligature alone). No significant difference was found between the three doses of Cimetidine used in this study.

### Histological Analysis

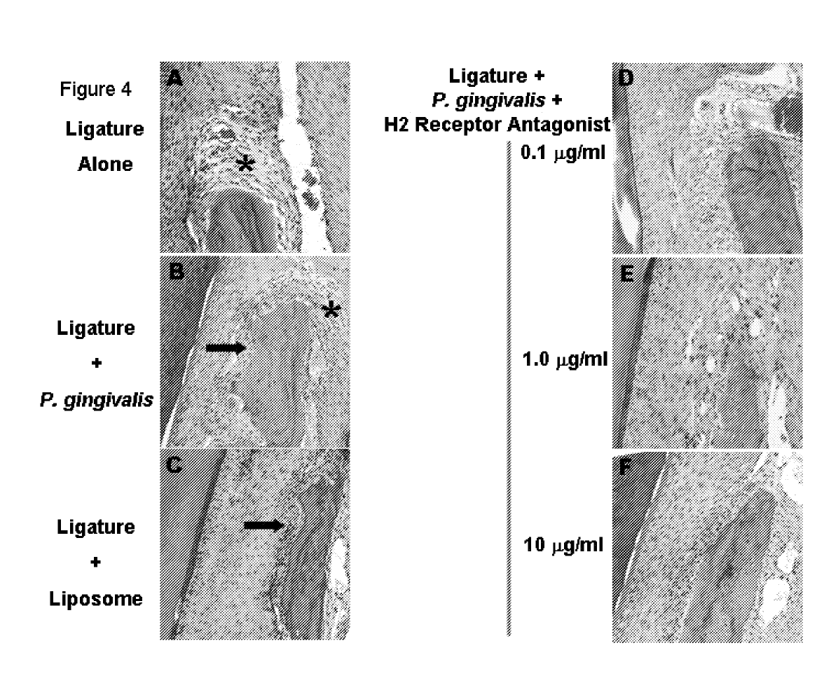

FIG. 4 shows the histological changes observed in response to different treatments. Hematoxylin and eosin stained sections of the ligated and diseased sites showed disrupted connective tissue layers with irregular fiber arrangement. Numerous blood vessels and inflammatory cells were localized adjacent to the basal layer in the connective tissue. Dense inflammatory infiltration spread to the lamina dura of the alveolar process bone, leading to evident bone destruction, and the alveolar borders were extremely ragged. The non-ligated sides showed healthy non-disrupted features. Ligature placement alone around the second pre-molars of rabbit mandible led to increased numbers of inflammatory cells (the presence of which is indicated by * on the pictures of FIG. 4) while neither bone loss nor any osteoclastic activity were visible (FIG. 4, Panel A). Local P. gingivalis administration in addition to ligature placement led to significant bone resorption as depicted by black arrows and increased inflammation (FIG. 4, Panel B). Liposome alone did not have any preventive or aggravating effect on the development of periodontitis (FIG. 4, Panel C).

All three doses of topical Cimetidine applications (0.1, 1.0, and 10 μg/mL) were found to prevent both bone loss and inflammatory changes in rabbits that received P. gingivalis and ligature placement (Groups D, E, and F). In the groups where Cimetidine, the H&E stained sections showed intact epithelium, dense, well-defined connective tissue fibers, less blood vessels and few numbers of inflammatory cells were observed in ligated sites (FIG. 4, Panels D-F). Regular alveolar bone borders in most areas and a few signs of alveolar bone resorption or resorptive lacunae were seen. The deposition of secondary bone on borders was also observed. No significant differences were found between the effects of different doses of Cimetidine used in this study.

### Osteoclastic Cell Activity

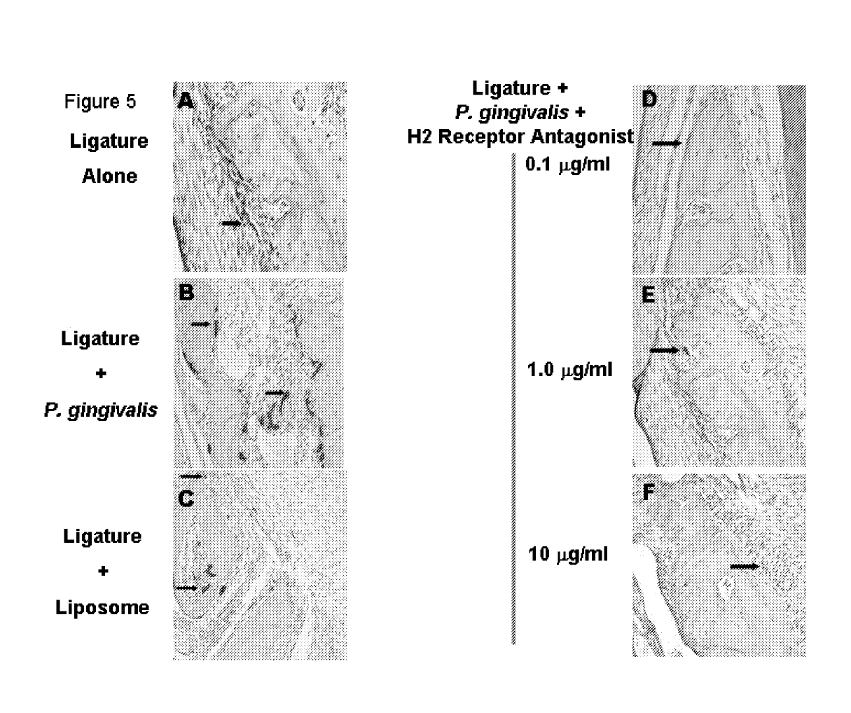

The TRAP stained sections of the ligated and diseased sites of the control group showed disrupted connective tissue and increased inflammatory cell infiltrate especially at the alveolar bone borders. Ligation alone did not lead to any increase in osteoclast numbers (FIG. 5, Panel A). The alveolar bone borders were found to be extremely ruffled with increased numbers of irregular shaped Howship's resorptive lacunae presenting osteoclast activity. Many multinucleated osteoclasts were observed on the resorptive areas (FIG. 5, Panel B).

In the vehicle group, liposome alone in addition to the experimental periodontitis did not prevent the osteoclastic activity (FIG. 5, Panel C). The TRAP stained sections of this group showed the same descriptive histology as the control group. The disrupted connective tissue and increased inflammatory cell infiltrate were obvious especially at the alveolar bone borders. The alveolar bone borders were extremely ruffled with increased numbers of irregular shaped Howship's resorptive lacunae presenting multinucleated osteoclastic activity.

In the Cimetidine groups, osteoclastic cells were either unidentifiable or present at few numbers (FIG. 5, Panel D-F). Although all three doses had a significant (p<0.05) preventive effect of one resorptive activity induced by periodontitis, there was a significant but gradual decrease in the number of osteoclasts and density with increasing dose. Overall, the TRAP stained sections showed intact epithelium, dense connective tissue layers with few blood vessels. Intact, regular, and well-defined alveolar bone borders were seen in most of areas, except for a few signs of osteoclasts and alveolar bone resorption. The deposition of secondary bone on borders was seen. No signs of multinucleated osteoclastic activity were seen.

### Histomorphometrical Analysis

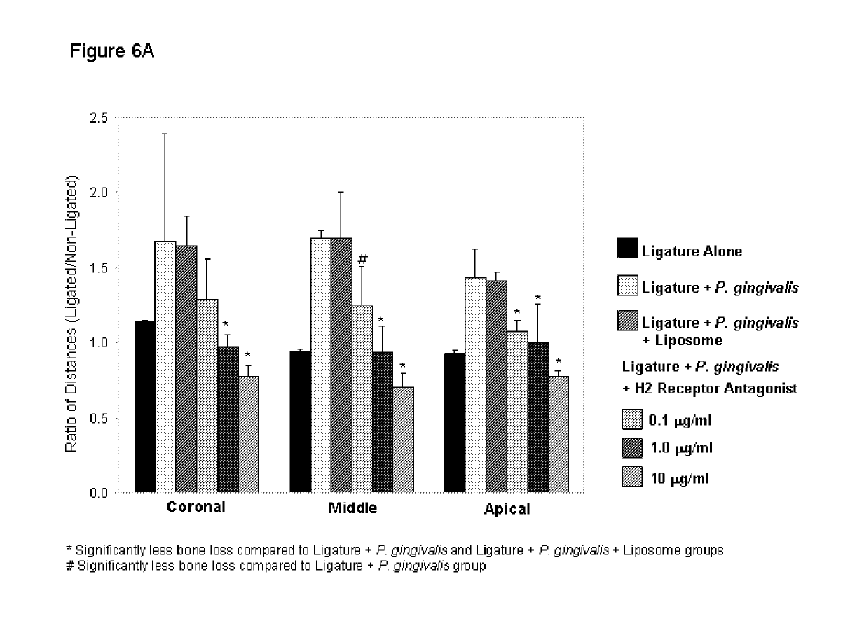

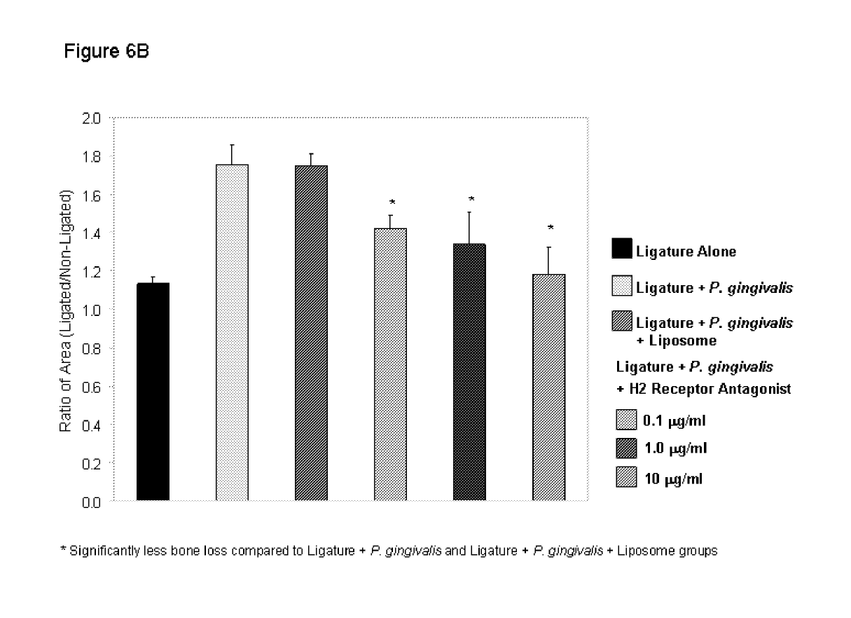

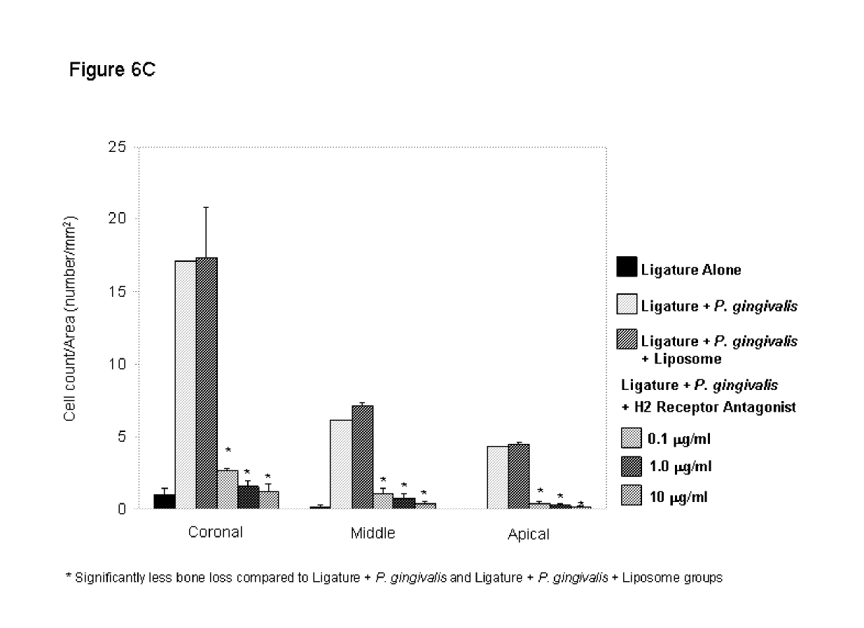

In order to quantitatively analyze periodontal disease progression in the rabbits treated with Cimetidine as compared to untreated ones, the mean value (±standard deviation) of linear distance and area were calculated for each group (FIG. 6). The linear distance was defined as the distance from the epithelium to the alveolar crest border at the three chosen levels, the tip, middle, and the base of the crest and was expressed as the ratio between the ligated and non-ligated sites. Likewise, area measurements were presented as the proportion of the total area at ligated to the non-ligated aspects of the teeth.

The ligated sites in the ligature+P. gingivalis and ligature+P. gingivalis+liposome groups showed significantly increased (p<0.05) distances compared to the Cimetidine-treated groups, which indicated the destruction of the alveolar bone crest due to the disease activity (FIG. 6A). The total area as well as the area of ligated side of the alveolar crest was significantly reduced in the control and vehicle group (p<0.05) (FIG. 6B).

The number of osteoclasts at the apical, middle, and coronal thirds of the root was another variable that was compared between the groups. The ligature+P. gingivalis and ligature+P. gingivalis+liposome groups presented markedly increased numbers of osteoclasts at all three levels with statistically significant values (p<0.05), whereas the Cimetidine groups showed comparable, nonsignificant values at the tip, middle and the base of the crest (p<0.05) (FIG. 6C).

### C. Discussion

The present study demonstrates that local administration of Cimetidine/NOVASOME®in three different dosages arrests tissue destruction and affects cell populations present in the inflammatory cell infiltrate associated with experimentally induced periodontitis in a rabbit animal model. The results of these histopathological and morphological observations showed that tissue change were induced by topical application of P. gingivalis and ligature placement. These changes were prevented by topical administration of H2 receptor antagonist (Cimetidine encapsulated into NOVASOME®) while simultaneous topical administration of P. gingivalis was continued. There were statistically significant (p<0.05) histomorphometric differences between the control, vehicle and cimetidine groups. The ligatured sites of the control and vehicle groups showed significant differences in the linear distances from the epithelium to the alveolar crest border at the three chosen levels—the apical, middle and coronal thirds −(p<0.05) as compared to the other three groups. The mean ratio of the linear distances of the ligated to non-ligated sites of the vehicle group was significantly higher when compared to the other three groups (p<0.05).

The present study histologically confirmed the relationship between alveolar bone loss and the presence of P. gingivalis. The cimetidine groups at three different doses ((0.1 μg/mL, 1.0 μg/mL, and 10.0 μg/mL) showed almost similar, insignificant (p>0.05) mean ratio values, indicating the preventive effect of the cimetidine against periodontal disease caused by P. gingivalis. The total area as well as the ligatured area of the alveolar crest was significantly reduced in the control and vehicle groups (p<0.05). The comparison of the total, the ligatured and the ratio of the ligatured/non-ligatured areas of the cimetidine groups showed no significant differences between the cimetidine groups (p>0.05).

Overall, these results support the concept that histamine, which has an immunomodulatory action, may be involved in the regulation of the local acute inflammatory responses in periodontal disease. Also, the findings of this study include histological evidence that treatment of periodontally diseased teeth with topically active cimetidine inhibits P. gingivalis-elicited leukocyte migration toward the site of infection and therefore arrests or prevents tissue destruction and affects cell populations present in the inflammatory cell infiltrate.

Histamine's effect on inflammation could be due to its direct or indirect effects on cells at early stages and seems to be receptor-regulated. While enhancing helper T cell type 1 (T_{H}1-type) responses via the H1 receptor, both T_{H}1 and T_{H}2 type responses are negatively regulated by H2 receptor activation (K. B. Hahm et al., Scand. J. Gastroenterol., 1995, 30: 265-271). Histamine's effect on neutrophil granulocytes has been well-documented and linked to inflammatory events. Histamine inhibits T-lymphocyte and natural killer cell-mediated cytotoxicity (B. E. Seligmann et al., J. Immunol., 1983, 130: 1902-1909). Histamine also depresses chemotaxis of neutrophils and the production of superoxide anion, hydrogen peroxide formation, degranulation of B-glucuronidase and lysozyme, and stimulated changes in membrane potential (B. E. Seligmann et al., J. Immunol., 1983, 130: 1902-1909). The effects of histamine on neutrophil motility are associated with increased levels of intracellular cAMP. In a series of in vitro experiments, it has been demonstrated that histamine at a range of 10 nM to 1 mM exerted a progressive and profound inhibition of neutrophil chemotaxis, an effect, which could be eliminated by an H2 receptor antagonist (R. Anderson et al., J. Immunol., 1977, 118: 1690-1696). These data suggest that H2 receptors may play a pivotal role in regulating histamine-mediated inflammatory reactions and multiple physiological events extending from gastric acid secretion to tissue inflammation (H. J. Nielsen et al., Arch. Surg., 1994, 129: 309-315). Indeed, treatment with H2 receptor antagonists has been shown to increase neutrophil chemotaxis (R. Anderson et al., J. Immunol., 1977, 118: 1690-1696; B. E. Seligmann et al., J. Immunol., 1983, 130: 1902-1909). Cimetidine reduces the superoxide (O₂·-) and hydrogen peroxide (H₂O₂) production of neutrophils in a dose-dependent manner (K. Mikawa et al., Anesth. Analg., 1999, 89: 218-224).

Histamine and H2 receptor antagonists are also recognized as modulators of B-cell and T-cell function via cell surface H2 receptor interactions. Specifically, histamine has been shown to directly inhibit B-cell production of immunoglobulin (IgG and IgM). This inhibition of B-cell antibody production by histamine can be blocked by treatment with cimetidine, which has also been shown to stimulate antibody production (M. Fujimoto and H. Kimata, Clin. Immunol. Immunopathol., 1994, 73: 96-102; W. B. Ershler et al., Clin. Immunol. Immunopathol., 1983, 26: 10-17; A. Kumar et al., Comp. Immunol. Microbiol. Infect. Dis., 1990, 13: 147-153). In addition, cimetidine treatment appears to modulate IgG subclass (enhanced IgG1 production) expression. H2 receptor antagonists are also widely recognized to modulate T-cell function through inhibition of suppressor T-lymphocyte activity, an increase in interleukin-2 production and enhancement of natural killer cell activity. Collectively, these observations suggest that H2 receptor antagonists may enhance host defense through both humoral and cellular pathways. Both cimetidine and metiamide, another H2 receptor antagonist, markedly influence the primary humoral antibody response of immunized normal cells in vitro. Optimum enhancement occurs in lower dosage (10 μg) on first day (H. Friedman et al., Proc. Sco. Exp. Biol., Med., 1982, 169: 222-225). Cimetidine influences certain IgG subclasses (enhanced IgG1 production) and IgM expression in vitro, however, route, timing and dosage of cimetidine administration are critical in modulating these effects (A. M. Badger et al., Immunology, 1983, 48: 151-155; Comp. Immunol. Microbiol. Infect. Dis., 1990, 13: 147-153). These variations in their effects might be due to their structural differences. Among all the H2 receptor antagonists (cimetidine, ranitidine, and famotidine), cimetidine has the strongest immunomodulating effect and only cimetidine augments the cytotoxicity and proliferative response of lyphocyte to mitogen (K. B. Hahm et al., Scand. J. Gastroenterol., 1995, 30: 265-271).

The aim of this study was to quantitatively analyze periodontal disease progression in rabbits treated with Cimetidine/NOVASOME® using histopathologic and histomorphometric analyses. The histomorphometrical analysis of the histological sections showed the preventive role of Cimetidine/NOVASOME® against periodontal disease. In fact, the present results showed significant alveolar bone loss at 6 weeks of the induction of experimental periodontitis. Increased multinucleated osteclastic cells with resorptive lacunae and inflammatory infiltrate dominated the pathological sections of the control and vehicle groups. Furthermore, numerous blood vessels and inflammatory cells were localized adjacent to the basal layer in the connective tissue. On the other hand, the cimetidine treated groups with three different dosages showed intact epithelium, dense, well defined connective tissue fibers, and scarce blood vessels, few numbers of inflammatory cells, with very regular bone borders. No signs of alveolar bone resorption and borders of secondary bone deposition were seen. The dose of cimetidine used in the present study were chosen empirically and the three groups of cimetidine showed comparable results. Thus, it appears that future studies will have to lower the dose to determine the minimal effective dose in animals or current doses could be used developing efficient medications in human disease models.

Therapeutic agents that are directed at modulation of various host mediators have shown significant promise for the management of adult periodontitis, and may be most appropriately indicated for individuals with substantial increased risk for periodontitis. The present study has provided histologic evidence confirming the role of a therapeutic host modulator agent via topical application of the H2 receptor antagonist Cimetidine in liposomes in the prevention of inflammatory cell infiltration, connective tissue destruction and bone loss in a rabbit periodontitis model. In conclusion, prospective data have been obtained suggesting that local cimetidine application can arrest the periodontal inflammation induced by P. gingivalis. Further, the evidence suggests that cimetidine could be used as a preventive agent in those subjects who are susceptible to periodontal disease. The findings of this study suggest that clinical therapeutic effect of local liposome-encapsulated cimetidine application in chronic periodontal conditions may be positive in humans, which may lead to discover new, effective and safer therapeutic applications to modulate host defense in response to resistant biofilms.

### Other Embodiments

Other embodiments of the invention will be apparent to those skilled in the art from a consideration of the specification or practice of the invention disclosed herein. It is intended that the specification and Examples be considered as exemplary only, with the true scope of the invention being indicated by the following claims.

What is claimed is:

1. A method for preventing or treating a periodontal disease in a subject, the method comprising a step of administering to the subject's oral cavity an effective amount of a liposome-encapsulated H2 antagonist.

2. The method of claim 1, wherein the step of administering comprises topically administering the encapsulated H2 antagonist.

3. The method of claim 1, wherein the subject is a human.

4. The method of claim 1, wherein the H2 antagonist is a compound selected from the group consisting of cimetidine, famotidine, nizatidine, and ranitidine.

5. The method of claim 1, wherein the H2 antagonist is encapsulated in a liposome selected from the group consisting of unilamellar liposome, multilamellar liposome, and paucilamellar liposome.

6. The method of claim 1, wherein the periodontal disease is gingivitis.

7. The method of claim 1, wherein the periodontal disease is periodontitis.

8. The method of claim 1, wherein the H2 antagonist is cimetidine.

9. The method of claim 8, wherein the step of administering comprises topically administering the encapsulated H2 antagonist.

10. The method of claim 9, wherein the H2 antagonist is encapsulated in a paucilamellar liposome.

11. The method of claim 10, wherein the periodontal disease is gingivitis.

12. The method of claim 10, wherein the periodontal disease is periodontitis.

13. The method of claim 10, wherein the method is a method for preventing the periodontal disease in the subject.

14. The method of claim 1, wherein the H2 antagonist is ranitidine.

15. The method of claim 14, wherein the step of administering comprises topically administering the encapsulated H2 antagonist.

16. The method of claim 15, wherein the H2 antagonist is encapsulated in a paucilamellar liposome.

17. The method of claim 16, wherein the periodontal disease is gingivitis.

18. The method of claim 16, wherein the periodontal disease is periodontitis.

19. The method of claim 16, wherein the method is a method for preventing the periodontal disease in the subject.
